(19) United States
(12) Patent Application Publication
(10) Pub. No.: US 2025/0049801 A1
(43) Pub. Date: Feb. 13, 2025

(54) PHARMACEUTICAL COMBINATIONS FOR USE IN THE TREATMENT OF NEOPLASTIC DISEASES

(71) Applicant: SILLAJEN, INC., Seoul (KR)

(72) Inventors: Heidi LANE, Allschwil (CH); Felix BACHMANN, Allschwil (CH); Karine LITHERLAND, Allschwil (CH); Paul MCSHEEHY, Allschwil (CH); Nicole FORSTER-GROSS, Allschwil (CH)

(21) Appl. No.: 18/717,562

(22) PCT Filed: Dec. 14, 2022
(86) PCT No.: PCT/KR2022/020390

§ 371 Date: Jun. 7, 2024

(30) Foreign Application Priority Data
Dec. 15, 2021 (EP) 21214940.5
Jun. 28, 2022 (EP) 22181675.4

(51) Int. Cl. A61K 31/519; A61K 31/337; A61P 35/00

(52) U.S. Cl. CPC A61K 31/519; A61K 31/337; A61P 35/00

(57) ABSTRACT

The present invention provides pharmaceutical combinations comprising (a) a TTK/PLK1 inhibitor (i.e. a compound of formula (I) or a pharmaceutically acceptable salt thereof as defend in the claims) and (b) a taxane anticancer drug or a pharmaceutically acceptable salt thereof (e.g. paclitaxel), as well as methods of using the combinations of the invention for the treatment of neoplastic diseases such as cancer.

### TECHNICAL FIELD

The present invention relates to pharmaceutical combinations comprising two active pharmaceutical compounds as described herein and methods of using the combinations of the invention in the treatment of neoplastic diseases, in particular cancer.

### BACKGROUND ART

WO 2015/155042 describes a recently discovered class of inhibitors of the threonine tyrosine kinase (TTK) for use in the treatment of cancer.

Paclitaxel belongs to the class of taxane anticancer drugs and is a well-known small molecule approved for the treatment of a number of cancer indications. It is commercialized under the brand name Taxol®.

There is an ongoing need for new effective treatment options for cancer patients. As demonstrated in the Examples below it has now surprisingly been found that the combination of a particular compound from WO 2015/155042 and paclitaxel provides multiple cures in cancer models.

### DISCLOSURE OF INVENTION

### Solution to Problem

### Summary of the Invention

In a first aspect the present invention provides a pharmaceutical combination comprising
(a) a compound of formula (I)

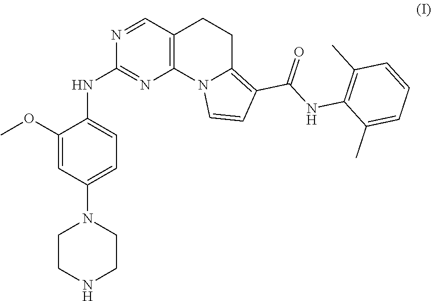 [SMILES: COc1cc(N2CCNCC2)ccc1Nc1ncc2c(n1)-n1ccc(C(=O)Nc3c(C)cccc3C)c1CC2]

or a pharmaceutically acceptable salt thereof;
and (b) a taxane anticancer drug or a pharmaceutically acceptable salt thereof (e.g. paclitaxel).

The compound of formula (I) is disclosed in WO 2015/155042 as Example 17.

It has also surprisingly been found that in addition to TTK inhibitory activity the compound of formula (I) also exhibits polo-like kinase 1 (PLK1) inhibition (see the Examples below). Both kinases collaborate in activating the mitotic spindle assembly checkpoint (SAC) at the kinetochore to regulate chromosome alignment and segregation before the cell can exit mitosis. Von Schubert et al., Cell Reports 2015, 12; 66-78 discloses that PLK1 and TTK (also known as MPS1) cooperatively regulate the spindle assembly checkpoint (SAC) in human cells. This potential for an enhanced effect from inhibition of both TTK and PLK1 is also alluded to in Dou et al., Plos ONE 2011, 6:4; e18793, which shows that some substrates of both kinases share a similar consensus motifs.

In contrast to TTK-specific inhibitors, the compound of formula (I) has a prolonged effect on TTK combined with a transient effect on PLK1 (see Examples below) leading to a more rapid disruption of the SAC that potentiates aberrant mitotic progression. Accordingly, the dual TTK/PLK1 inhibitory activity gives the compound of formula (I) a unique profile and differentiates it from other molecules which show TTK inhibitory activity without any appreciable levels of PLK1 inhibitory activity.

In a further aspect the invention provides a method for treating a neoplastic disease in a subject in need thereof comprising administering to the subject a therapeutically effective amount of the pharmaceutical combination of the invention.

In a further aspect the invention provides a method for treating a neoplastic disease in a subject in need thereof comprising administering to the subject a therapeutically effective amount of the compound of formula (I) or a pharmaceutically acceptable salt thereof, wherein said subject is undergoing or will undergo treatment with a taxane anticancer drug or a pharmaceutically acceptable salt thereof (e.g. paclitaxel).

In a further aspect the invention provides a method for treating a neoplastic disease in a subject in need thereof comprising administering to the subject a therapeutically effective amount of a taxane anticancer drug or a pharmaceutically acceptable salt thereof (e.g. paclitaxel), wherein said subject is undergoing or will undergo treatment with the compound of formula (I) or a pharmaceutically acceptable salt thereof.

In a further aspect the invention provides the pharmaceutical combination of the invention for use in the treatment of a neoplastic disease in a subject.

In a further aspect the invention provides a compound of formula (I) or a pharmaceutically acceptable salt thereof for use in combination with a taxane anticancer drug or a pharmaceutically acceptable salt thereof (e.g. paclitaxel) for the treatment of a neoplastic disease in a subject.

In a further aspect the invention provides a taxane anticancer drug or a pharmaceutically acceptable salt thereof (e.g. paclitaxel) for use in combination with a compound of formula (I) or a pharmaceutically acceptable salt thereof for the treatment of a neoplastic disease in a subject.

In a further aspect the invention provides use of the pharmaceutical combination of the invention in the preparation of single-agent medicaments or as a combined medicament for the treatment of a neoplastic disease in a subject.

In a further aspect the invention provides use of a compound of formula (I) or a pharmaceutically acceptable salt thereof in the preparation of a single-agent medicament for use in combination with a taxane anticancer drug or a pharmaceutically acceptable salt thereof (e.g. paclitaxel), or in the preparation of a combined medicament with a taxane anticancer drug or a pharmaceutically acceptable salt thereof (e.g. paclitaxel), for the treatment of a neoplastic disease in a subject.

In a further aspect the invention provides use of a taxane anticancer drug or a pharmaceutically acceptable salt thereof (e.g. paclitaxel) in the preparation of a single-agent medicament for use in combination with a compound of formula (I) or a pharmaceutically acceptable salt thereof or in the preparation of a combined medicament with the compound of formula (I) or a pharmaceutically acceptable salt thereof, for the treatment of a neoplastic disease in a subject.

Neoplastic diseases for treatment by combinations of the invention are described below, and are in particular contemplated for treatment of cancer, and in particular for human subjects.

Additional aspects and embodiments of the invention are described in more detail below.

### BRIEF DESCRIPTION OF FIGURES

FIG. 1: FIG. 1 shows the effect of monotherapies of the compound of formula (I) and paclitaxel (PTX) and their combination in mice bearing the subcutaneous patient derived xenograft (PDX) tumor model BR1282 from the first PDX study. (A) and (B) show results of growth of the BR1282 tumor under the different treatments (A, mean±SEM) and a comparison of the different rates of growth of each tumor for each treatment group (B, mean±SD), where *** indicates p<0.001 compared to the vehicle and both monotherapies (the combination groups were not significantly different to each other; p=0.7). Note: on day 21, one mouse died in the compound of formula (I)-monotherapy group (considered non-drug-related), and on day 28, one mouse was culled due to >20% body weight loss in the combination 24 hour group. (C) and (D) show the percentage change in body weight under the different treatments (C, mean±SEM) and a comparison at day 16 when the vehicle group was culled, where *p<0.05 (D). Statistical analysis used a one-way ANOVA with Holm-Sidak post-hoc. N=8 mice/group. The control mice received both vehicles using a 4 h gap between administrations, i.e. the vehicle of the compound of formula (I) and the vehicle of paclitaxel.

FIG. 2: FIG. 2 shows a comparison of individual tumor growth in the 5 treatment groups in the subcutaneous PDX tumor model BR1282 from the first PDX study shown in FIG. 1. N=8 mice/group. (A) shows the vehicle control group, (B) shows the group receiving the compound of formula (I) alone, (C) shows the group receiving paclitaxel alone, (D) shows the group receiving the combination therapy in which the compound of formula (I) was administered four hours after paclitaxel when both compounds were administered on the same day, (E) shows the group receiving the combination therapy in which the compound of formula (I) was administered twenty four hours after paclitaxel when both compounds were administered on consecutive days.

FIG. 3: FIG. 3 shows Hematoxylin and Eosin (H&E) stains of mouse skin prepared on day 97 from the first PDX study. (A) shows mouse skin in the combination 24 hour group showing residual tumor (indicated by the star). Calcification (indicated by the arrow) is also visible. (B) shows mouse skin in the combination 24 hour group showing a complete absence of residual tumor.

FIG. 4: FIG. 4 shows the effect of monotherapies and their combination in mice bearing the subcutaneous PDX tumor model BR1282 from the second PDX study. (A) and (B) show results of growth of the BR1282 tumor under the different treatments (A, mean±SEM) and a comparison of the different rates of growth of each tumor for each treatment group up to the day of culling or day 51 which was one week after the final treatment (B, mean±SD), where *** indicates p<0.001, ** indicates p<0.01 and * indicates p<0.05 compared to monotherapy of compound of formula (I). All combination groups showed p<0.001 compared to the vehicle and paclitaxel monotherapy (the combination groups were not significantly different to each other; p=0.957-0.998). Note: on day 23, one mouse was culled due to >20% body weight loss in the combination group with a 4 hour gap (i.e. the treatment group indicated as PTX 15 mg/kg; Formula (I) 8 mg/kg 2qw (4 h gap)). (C) and (D) show the body weight change in percent under the different treatments (C, mean±SEM) and a comparison at day 13 when the vehicle group was culled, where *p<0.05, ** p<0.01 and *** p<0.001 (D). Statistical analysis used a one-way ANOVA with Holm-Sidak post-hoc. N=8 mice/group. The control mice received both vehicles using a 4 h gap between administration, i.e. the vehicle of the compound of formula (I) and the vehicle of paclitaxel.

FIG. 5: FIG. 5 shows a comparison of individual tumor growth in the 9 treatment groups in the subcutaneous PDX tumor model BR1282 from the second PDX study shown in FIG. 4. N=8 mice/group. (A) shows the vehicle control group, (B) shows the group receiving the compound of formula (I) alone, (C) shows the group receiving paclitaxel alone, (D) shows the group receiving the combination therapy in which the compound of formula (I) was administered two hours after paclitaxel when both compounds were administered on the same day, (E) shows the group receiving the combination therapy in which the compound of formula (I) was administered four hours after paclitaxel when both compounds were administered on the same day, (F) shows the group receiving the combination therapy in which the compound of formula (I) was administered twenty four hours after paclitaxel when both compounds were administered on consecutive days, (G) shows the group receiving the combination therapy in which the compound of formula (I) was administered as a single dose four hours after paclitaxel, (H) shows the group receiving the combination therapy in which the compound of formula (I) was administered four hours before paclitaxel when both compounds were administered on the same day.

FIG. 6: FIG. 6 shows the results of experiments to determine if there was a significant drug-drug interaction (DDI) between the compound of formula (I) and paclitaxel. (A) shows the influence of paclitaxel on the compound of formula (I). Non tumor-bearing mice (n=3 for each group) were treated once with the compound of formula (I) alone (8 mg/kg, intravenous (i.v.)) or with the compound of formula (I) (8 mg/kg, i.v.) one hour after administration of paclitaxel (15 mg/kg, i.v.). Results show the mean±SD (n=3) concentration-time curves for the different treatment regimens. (B) shows the influence of the compound of formula (I) on paclitaxel. Non tumor-bearing mice (n=3 for each group) were treated once with paclitaxel alone (15 mg/kg, i.v.) or with paclitaxel (15 mg/kg, i.v.) one hour before administration of the compound of formula (I) (8 mg/kg, i.v.). Results show the mean±SD (n=3) concentration-time curves for the different treatment regimens.

### DETAILED DESCRIPTION OF THE INVENTION

### Definitions

Certain terms used herein are described below. Unless defined otherwise, all technical and scientific terms used herein have the same meaning as is commonly understood by one of skill in the art to which this invention belongs.

The term “combination”, “therapeutic combination”, or “pharmaceutical combination” are interchangeable terms and refer to either a fixed combination or a non-fixed combination.

The term “combination therapy” refers to the administration of two or more therapeutic agents to treat a therapeutic condition or disorder described in the present disclosure. Such administration encompasses co-administration of these therapeutic agents in a substantially simultaneous manner as well as use of each type of therapeutic agent in a sequential and/or separate manner (e.g. according to different administration routes), either at approximately the same time or at different times, e.g. according to different dosage regimens, examples of which are described herein. When the therapeutic agents are administered sequentially and/or separately the dosing schedules will be such that there is a therapeutic interaction between the therapeutic agents within the patient's body and/or that a therapeutic effect resulting from the first therapeutic agent is present when the second therapeutic agent is administered. For example, when the agents are administered according to cyclic treatment schedules, the cyclic treatment schedules may overlap, or when one therapeutic agent is administered according to a continuous dosing schedule and the second according to a cyclic schedule, then at least one dose from the agent administered according to the continuous schedule will occur during the treatment cycle of the other therapeutic agent. Usually there will be at least one interval of no more than seven days between a dose of one of the therapeutic agents and a dose of the other therapeutic agent when both therapeutic agents are administered according to cyclic treatment schedules.

The term “pharmaceutical composition” is defined herein to refer to a solid or liquid formulation containing at least one therapeutic agent to be administered to a patient, optionally with one or more pharmaceutically acceptable excipients, in order to treat a particular disease or condition affecting the patient.

The term “pharmaceutically acceptable” as used herein refers to items such as compounds, materials, compositions and/or dosage forms, which are, within the scope of sound medical judgment, suitable for contact with the tissues of a human, without excessive toxicity or other complications commensurate with a reasonable benefit/risk ratio.

The terms “fixed combination”, “fixed dose”, “single formulation” and “combined medicament” as used herein refers to a single dosage form formulated to deliver an amount, which is jointly therapeutically effective for the treatment of neoplastic diseases, of both therapeutic agents to a patient. The single dosage form is designed to deliver an amount of each of the therapeutic agents, along with any pharmaceutically acceptable carriers or excipients.

The term “non-fixed combination,”, “kit”, “separate formulations” and “single-agent medicaments” means that the active ingredients are formulated as separate entities to allow administration to a patient either simultaneously, sequentially or separately, wherein such administration provides therapeutically effective levels of the two compounds in the body of the patient.

The term “patient” refers to a human presenting themselves for therapeutic treatment.

The term “subject” refers to a mammal and preferably refers to a patient.

The term “treatment,” as used herein in the context of treating a disease in a subject pertains generally to treatment and therapy in which some desired therapeutic effect is achieved, for example one or more of the following: the inhibition of the progress of the disease, a reduction in the rate of progress, a halt in the rate of progress, a prevention of the progression of the disease, alleviation of symptoms of the disease, amelioration of disease, and cure of the disease. For example, treatment can be the diminishment of one or several symptoms of a disorder or complete eradication of a disorder. Within the meaning of the present disclosure, the term “treat” also denotes to arrest, delay the onset (i.e. the period prior to clinical manifestation of a disease) and/or reduce the risk of developing or worsening of a disease.

The term “prevent”, “preventing” or “prevention” as used herein comprises the prevention of at least one symptom associated with or caused by disease being prevented.

The term “pharmaceutically effective amount,” “therapeutically effective amount,” or “clinically effective amount” is an amount sufficient to provide an observable or clinically significant improvement over the baseline clinically observable signs and symptoms of the disease treated, e.g. commensurate with a reasonable benefit/risk ratio, when administered in accordance with a desired treatment regimen. The skilled person will understand that the therapeutically effective amount of an agent for use in combination therapy may be lower than the amount required to provide a therapeutic effect when using the agent as a monotherapy.

The term “about” means a variation of no more than 10% of the relevant figure. In some embodiments the term “about” means a variation of no more than 5% of the relevant figure.

For the avoidance of doubt, where a range is provided (e.g. 5 mg to 480 mg) the range includes the stated upper limit (480 mg) and lower limit (5 mg) of the range.

### Compounds of Formula (I)

In some embodiments the compound of formula (I) is/is used as the free base. In other embodiments the compound of formula (I) is used as a pharmaceutically acceptable salt.

Pharmaceutically acceptable salts of the compound of formula (I) may be acid addition salts. Salts are formed e.g. with organic or inorganic acids from compounds of formula (I). Pharmaceutically acceptable salts are within the common general knowledge of the person skilled in the art. Pharmaceutically acceptable salts may include more than one molecule or ion of the corresponding acid.

The compounds of formula (I) and pharmaceutically acceptable salts thereof may be solvated, especially hydrated. Solvation and/or hydration may take place during the preparation process.

Compounds of formula (I) and pharmaceutically acceptable salts thereof may be synthesized as described in WO 2015/155042, in particular on pages 17 to 19 which are hereby incorporated by reference, and as described in Example 17 on page 49 of WO 2015/155042, which is also hereby incorporated by reference, including the reference in Example 17 to Example 9, Intermediate H and Example 1.

### Taxane Anticancer Drugs

Taxanes are a well-known class of anticancer drugs, which includes e.g. paclitaxel (sold under the tradename Taxol®), docetaxel (sold under the tradename Taxotere®) and cabazitel (sold under the tradename Jevtana®). Other taxanes are in clinical development, such as larotaxel, milataxel, ortataxel and tesetaxel (see e.g. Ojima et al. Expert Opin. Ther. Pat. 2016, 26 (1): 1-20). The principal mechanism of action of the taxane class of anticancer drugs is the disruption of microtubule function. Taxanes stabilize guanosine diphosphate (GDP)-bound tubulin in the microtubule. Stabilization of the microtubules thereby inhibits the process of cell division.

In some embodiments the taxane anticancer drug or a pharmaceutically acceptable salt thereof is paclitaxel, docetaxel, cabazitaxel, larotaxel, milataxel, ortataxel or tesetaxel, including pharmaceutically acceptable salts thereof. In some embodiments the taxane anticancer drug or a pharmaceutically acceptable salt thereof is paclitaxel, docetaxel or cabazitel or a pharmaceutically acceptable salt thereof. Preferably the taxane anticancer drug or a pharmaceutically acceptable salt thereof is paclitaxel or a pharmaceutically acceptable salt thereof, and more preferably paclitaxel (e.g. as sold under the brand name Taxol®).

### Diseases

The pharmaceutical combinations of the invention may be used to treat neoplastic diseases by administration of the combinations of the invention, e.g. to inhibit the protein kinase TTK (compound of formula (I) or a pharmaceutically acceptable salt thereof) and stabilize microtubules (taxane anticancer drug, e.g. paclitaxel). In addition in view of the observation that compounds of formula (I) additionally have PLK1 inhibitory activity the neoplastic disease may be one which is treatable by inhibition of PLK1 in addition to a treatment with a taxane anticancer drug (e.g. paclitaxel) and a TTK inhibitor (e.g. the compound of formula (I)).

In addition, the pharmaceutical combinations of the invention may be used to treat a cancer at any clinical stage or pathological grade (e.g. tumor stage I, tumor stage II, tumor stage III, tumor stage IV) or treatment settings (e.g. preventative, adjuvant, neoadjuvant, therapeutic including palliative treatment). The pharmaceutical combinations of the invention may be for use in slowing, delaying or stopping cancer progression or cancer growth or increasing the overall survival time or the cancer-progression-free survival time or the time to progression of a cancer or improving or maintaining the subject's (e.g. patient's) quality of life or functional status. The pharmaceutical combinations of the invention may also be used in post-therapy recovery from cancer. The pharmaceutical combinations of the invention may be used in the treatment of metastatic cancer.

For example, the pharmaceutical combinations of the invention may be used for (i) reducing the number of cancer cells; (ii) reducing tumor volume; (iii) increasing tumor regression rate; (iv) reducing or slowing cancer cell infiltration into peripheral organs; (v) reducing or slowing tumor metastasis; (vi) reducing or inhibiting tumor growth; (vii) preventing or delaying occurrence and/or recurrence of the cancer and/or extends disease- or tumor-free survival time; (viii) increasing overall survival time; (ix) reducing the frequency of treatment; and/or (x) relieving one or more of symptoms associated with the cancer.

As mentioned above, the pharmaceutical combinations of the invention may be used for the treatment of neoplastic diseases. Examples of neoplastic diseases include, but are not limited to, epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, nacvi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, miscellaneous myeloproliferative disorders, lymphoproliferative disorders and myelodysplastic syndromes.

In some embodiments the neoplastic disease is cancer. Examples of cancers in terms of the organs and parts of the body affected include, but are not limited to, the brain, breast (including triple negative breast cancer and luminal B breast cancer), cervix, ovaries, colon, rectum (including colon and rectum i.e. colorectal cancer) lung (including small cell lung cancer, non-small cell lung cancer, large cell lung cancer and mesothelioma), endocrine system, bone, adrenal gland, thymus, liver, stomach, intestine (including gastric cancer), pancreas, bone marrow, hematological malignancies (such as lymphoma, leukemia, myeloma or lymphoid malignancies), bile duct, bladder, urinary tract, kidneys, skin, thyroid, head, neck, prostate and testis.

In some embodiments the neoplastic disease is a cancer selected from breast cancer (including triple negative breast cancer and luminal B breast cancer), gastric cancer, colorectal cancer, liver cancer (including hepatocellular cancer), endometrial cancer, ovarian cancer, esophageal cancer, lung cancer (including non-small cell lung cancer), Kaposi's sarcoma, cervical cancer, pancreatic cancer, melanoma, prostate cancer, bladder cancer and leukemia, e.g. acute myeloid leukemia (AML) (including Complex Karyotype AML).

In some embodiments the neoplastic disease is breast cancer.

In some embodiments the neoplastic disease is triple negative breast cancer.

In some embodiments the neoplastic disease is luminal B breast cancer.

In some embodiments the neoplastic disease is gastric cancer.

In some embodiments the neoplastic disease is colorectal cancer.

In some embodiments the neoplastic disease is hepatocellular cancer.

In some embodiments the neoplastic disease is endometrial cancer.

In some embodiments the neoplastic disease is acute myeloid leukemia (AML) (including Complex Karyotype AML).

The cancer may be a primary tumor and/or metastases. The cancer may be derived from a solid or liquid (e.g. hematological or intraperitoneal) tumor. In some embodiments the neoplastic disease (e.g. cancer) to be treated is a tumor, e.g. a solid tumor.

### Administration

Administration of the pharmaceutical combinations of the invention includes administration of the combination as a single formulation, as well as administration of the individual agents of the combination as separate formulations. Preferably the compound of formula (I) or a pharmaceutically acceptable derivative thereof and the taxane anticancer drug or a pharmaceutically acceptable salt thereof are comprised in separate formulations. Preferably the compound of formula (I) or a pharmaceutically acceptable salt thereof and the taxane anticancer drug or a pharmaceutically acceptable salt thereof (e.g. paclitaxel) are administered to a subject (preferably a human) intravenously.

In some embodiments, the combination of the invention is used for the treatment of cancer in a subject comprising administering to the subject a combination therapy, comprising a therapeutically effective amount of the compound of formula (I) or a pharmaceutically acceptable salt thereof and a therapeutically effective amount of a taxane anticancer drug or a pharmaceutically acceptable salt thereof (e.g. paclitaxel). These compounds are administered at therapeutically effective dosages, which when combined provide a beneficial effect e.g. as described herein. The skilled person will understand that therapeutically effective dosages for use in combination therapy may be lower than the dosages required to provide a therapeutic effect when using either agent as a monotherapy.

The administration of a pharmaceutical combination of the invention may result not only in a beneficial effect, e.g. a synergistic effect, e.g. with regard to alleviating, delaying progression of or inhibiting the symptoms, but may also result in further beneficial effects, e.g. fewer side-effects, more durable therapeutic effect, an improved quality of life and/or a decreased morbidity, compared with a monotherapy applying only one of the pharmaceutically therapeutic agents used in the combination of the invention. It may also be the case that lower doses of the therapeutic agents of the combination of the invention can be used, for example, such that the dosages may not only often be smaller, but also may be applied less frequently, or can be used in order to diminish the incidence of side-effects observed with one of the combination partners alone.

In some embodiments, the combination provided herein may display a synergistic effect. The term “synergistic effect” as used herein, refers to action of the two agents, namely the compound of formula (I) or a pharmaceutically acceptable salt thereof and a taxane anticancer drug or a pharmaceutically acceptable salt thereof (e.g. paclitaxel), to produce a therapeutic effect, e.g. slowing the progression of the disease or symptoms thereof, which is greater than the addition of the same therapeutic effect of each drug administered on its own. Generally, in determining a synergistic interaction between one or more components, the optimum range for the effect and absolute dose ranges of each component for the effect may be definitively measured by administration of the components over different w/w ratio ranges and doses to subjects (e.g. patients) in need of treatment. For humans, the complexity and cost of carrying out clinical studies on patients may render impractical the use of this form of testing as a primary model for synergy. However, the observation of synergy in certain experiments can be predictive of the effect in other species, and animal models may be used to further quantify a synergistic effect. The results of such studies can also be used to predict effective dose ratio ranges and the absolute doses and plasma concentrations, e.g. as illustrated in the Examples below.

In further embodiments, the present invention provides a synergistic combination for administration to humans comprising the pharmaceutical combination of the invention, where the dose range of each component corresponds to the synergistic ranges, e.g. as indicated in a suitable tumor model or clinical study.

The combinations of the present invention can be used in long-term therapy or as an adjuvant therapy in the context of other treatment strategies, as described above. Other possible treatments are therapy to maintain the subject's (e.g. patient's) status after tumor regression, or even preventive therapy, for example in subjects (e.g. patients) at risk.

The methods according to the invention may comprise (i) administration of the compound of formula (I) in free or pharmaceutically acceptable salt form and (ii) administration of the taxane anticancer drug or a pharmaceutically acceptable salt thereof (e.g. paclitaxel) in free or pharmaceutically acceptable salt form simultaneously, sequentially or separately in any order, in jointly therapeutically effective amounts, e.g. in synergistically effective amounts, e.g. in continuous or cyclic dosing schedules, e.g. corresponding to the amounts described herein. The individual combination partners of the combination of the invention may be administered separately at different times during the course of therapy or concurrently. The invention is therefore to be understood as embracing all such treatment regimens and the term “administering” is to be interpreted accordingly. Examples of treatment regimens for use with the invention are described in detail below.

The compound of formula (I) or a pharmaceutically acceptable salt thereof and the taxane anticancer drug or a pharmaceutically acceptable salt thereof (e.g. paclitaxel) may be administered according to the same treatment schedule or may be administered according to independent treatment schedules. The treatment schedules may be cyclic or continuous.

A cyclic treatment schedule is defined by a repeated dosing schedule wherein the repeated element (a cycle) has a specific duration and wherein doses are administered on specific days within the cycle. A cycle may incorporate a period, usually at the end of the cycle, in which there is no administration (a “rest period”), e.g. to allow a period for recovery. A treatment cycle may be, e.g. 7 days, 14 days, 21 days, 28 days or longer. A continuous treatment schedule is a regular dosing schedule, which does not incorporate rest periods (i.e. periods that are longer than the regular interval between the doses). For example, doses may be administered once per day, twice per day, once every two days, once every three days etc. The treatment schedule, whether cyclic or continuous may be continued for as long as required (an “open-end treatment”) e.g. as long as the subject (e.g. patient) is receiving benefit judged by a physician overseeing the treatment.

When the compound of formula (I) or a pharmaceutically acceptable salt thereof and the taxane anticancer drug or a pharmaceutically acceptable salt thereof (e.g. paclitaxel) are administered according to independent treatment schedules, the treatment schedules may both be cyclic, or one may be cyclic and the other may be continuous. When both treatment schedules are cyclic, the cycles of the two treatment schedules may be of the same duration or may be of different duration, and they may start on the same day or may start on different days. When the cycles are of the same duration the treatment cycles of both drugs will usually start on the same day.

In some embodiments the compound of formula (I) or a pharmaceutically acceptable salt thereof and the taxane anticancer drug or a pharmaceutically acceptable salt thereof (e.g. paclitaxel) are administered according to cyclic treatment schedules which may be of the same duration, e.g. four weeks/28 days, with the respective treatment cycles usually starting on the same day (i.e. the same treatment cycle).

In some embodiments, the compound of formula (I) or a pharmaceutically acceptable salt thereof and the taxane anticancer drug or a pharmaceutically acceptable salt thereof (e.g. paclitaxel) are administered (e.g. intravenously) according to a four week treatment cycle, wherein the compound of formula (I) or a pharmaceutically acceptable salt thereof is administered in week one of the treatment cycle, e.g. on day 1, followed by three rest weeks and the taxane anticancer drug or a pharmaceutically acceptable salt thereof (e.g. paclitaxel) is administered in each week during the first three weeks of the treatment cycle followed by a rest week, e.g. on days 1, 8 and 15. Preferably the compound of formula (I) or a pharmaceutically acceptable salt thereof and the taxane anticancer drug or a pharmaceutically acceptable salt thereof (e.g. paclitaxel) are administered to the subject according to the same four week treatment cycle (i.e. the treatment cycles start on the same day).

In some embodiments, the compound of formula (I) or a pharmaceutically acceptable salt thereof and the taxane anticancer drug or a pharmaceutically acceptable salt thereof (e.g. paclitaxel) are administered (e.g. intravenously) according to a four week treatment cycle, wherein the compound of formula (I) or a pharmaceutically acceptable salt thereof is administered in week one and in week three of the treatment cycle, e.g. on day 1 and day 15, with weeks two and four being rest weeks, and the taxane anticancer drug or a pharmaceutically acceptable salt thereof (e.g. paclitaxel) is administered in each week during the first three weeks of the treatment cycle followed by a rest week, e.g. on days 1, 8 and 15. Preferably the compound of formula (I) or a pharmaceutically acceptable salt thereof and the taxane anticancer drug or a pharmaceutically acceptable salt thereof (e.g. paclitaxel) are administered to the subject according to the same four week treatment cycle (i.e. the treatment cycles start on the same day).

In some embodiments, the compound of formula (I) or a pharmaceutically acceptable salt thereof and the taxane anticancer drug or a pharmaceutically acceptable salt thereof (e.g. paclitaxel) are administered (e.g. intravenously) according a four week treatment cycle, wherein the compound of formula (I) or a pharmaceutically acceptable salt thereof is administered in each week during the first three weeks of the treatment cycle, e.g. on days 1, 8 and 15, followed by a rest week, and the taxane anticancer drug or a pharmaceutically acceptable salt thereof (e.g. paclitaxel) is administered in each week during the first three weeks of the treatment cycle followed by a rest week, e.g. on days 1, 8 and 15. Preferably the compound of formula (I) or a pharmaceutically acceptable salt thereof and the taxane anticancer drug or a pharmaceutically acceptable salt thereof (e.g. paclitaxel) are administered to the subject according to the same four week treatment cycle (i.e. the treatment cycles start on the same day).

Effective dosages of each of the combination partners employed in the combinations of the invention may vary depending on the pharmaceutical composition employed, the mode of administration, the condition being treated, and the severity of the condition being treated. Thus, the dosage regimen of the combination of the invention is selected in accordance with a variety of factors including the route of administration and the renal and hepatic function of the subject (e.g. patient).

The optimum ratios, individual and combined dosages, and concentrations of the combination partners of the pharmaceutical combination of the invention that yield efficacy without toxicity are based on the kinetics of the therapeutic agents' availability to target sites. They may be established using routine clinical testing and procedures that are well known in the art and will depend upon a variety of factors, such as the mode of administration, the condition being treated and the severity of the condition being treated, as well as the age, body weight, general health, gender and diet of the individual and other medications the individual is taking. Likewise, frequency of dosage may vary depending on the compound used and the particular condition to be treated. Subjects (e.g. patients) may generally be monitored for therapeutic effectiveness using assays suitable for the condition being treated, which will be familiar to those of ordinary skill in the art.

In some embodiments the molar ratio of the taxane anticancer drug or a pharmaceutically acceptable salt thereof (e.g. paclitaxel) to the compound of formula (I) or a pharmaceutically acceptable salt thereof per weekly dose in the combinations of the invention may be e.g. about 0.1:1 to about 18.8:1, e.g. about 0.1:1 to about 16.7:1, e.g. about 0.2:1 to about 16.7:1, e.g. about 0.3:1 to about 2.3:1, e.g. about 0.3:1 to about 2.1:1, e.g. about 0.3:1 to about 0.7:1, e.g. about 0.3:1 to about 0.6:1, e.g. about 0.3:1 to about 0.5:1, e.g. about 0.4:1 to about 2.1:1, e.g. about 0.4:1 to about 1.2:1, e.g. about 0.4:1 to about 1.0:1, e.g. about 0.4:1 to about 0.5:1, e.g. about 0.5:1 to about 1.0:1, e.g. about 0.5:1 to about 0.9:1, e.g. about 0.6:1 to about 0.9:1. In some embodiments the molar ratio of the taxane anticancer drug or a pharmaceutically acceptable salt thereof (e.g. paclitaxel) to the compound of formula (I) or a pharmaceutically acceptable salt thereof per weekly dose is about 0.4:1 to about 2.1:1.

In some embodiments the molar ratio of the taxane anticancer drug or a pharmaceutically acceptable salt thereof (e.g. paclitaxel) to the compound of formula (I) or a pharmaceutically acceptable salt thereof per weekly dose may be e.g. up to about 18.8:1, e.g. up to about 16.7:1, e.g. up to about 2.3:1, e.g. up to about 2.1:1, e.g. up to about 1.2:1, e.g. up to about 1.0:1, e.g. up to about 0.9:1, e.g. up to about 0.7:1, e.g. up to about 0.6:1, e.g. up to about 0.5:1. In some embodiments the molar ratio of the taxane anticancer drug or a pharmaceutically acceptable salt thereof (e.g. paclitaxel) to the compound of formula (I) or a pharmaceutically acceptable salt thereof per weekly dose is up to about 2.1:1.

In some embodiments the molar ratio of the taxane anticancer drug or a pharmaceutically acceptable salt thereof (e.g. paclitaxel) to the compound of formula (I) or a pharmaceutically acceptable salt thereof per weekly dose may be e.g. at least about 0.1:1, e.g. at least about 0.2:1, e.g. at least about 0.3:1, e.g. at least about 0.4:1, e.g. at least about 0.5:1, e.g. at least about 0.6:1. In some embodiments the molar ratio of the taxane anticancer drug or a pharmaceutically acceptable salt thereof to the compound of formula (I) or a pharmaceutically acceptable salt thereof per weekly dose is at least about 0.4:1.

The following examples of dosages are for humans. The doses of compound of formula (I) are given as mg per person irrespective of body weight or body surface area (BSA). Dosages of the compound of formula (I) as given below, including in Tables A, B and C refer to dosages of the free base. The dosages also apply to pharmaceutically acceptable salts of the compound of formula (I), except that when a pharmaceutically acceptable salt of the compound of formula (I) is used the stated mg dosage amount should be adjusted (i.e. increased) so that the molar amount of the pharmaceutically acceptable salt of the compound of formula (I) to be dosed is the same as the molar amount of the free base as given below. For example, a statement that the (human) weekly dosage amount of the compound of formula (I) is about 5 mg to about 480 mg in weeks when administered means that the compound of formula (I) or a pharmaceutically acceptable salt thereof is administered to a patient at a dose corresponding to the mole equivalent of about 5 mg to about 480 mg of the free base of the compound of formula (I) per week in weeks when administered.

The doses of paclitaxel are given as mg/m² of BSA and refer to the free base. Likewise, the stated dosages of paclitaxel also apply to pharmaceutically acceptable salts of paclitaxel except that when a pharmaceutically salt of paclitaxel is used the stated mg/m² dosage amount should be adjusted (i.e. increased) so that the molar amount of paclitaxel to be dosed is the same as the molar amount of the free base. Paclitaxel is normally used as the free base.

In some embodiments the (human) weekly dosage amount of the compound of formula (I) is about 5 mg to about 480 mg in weeks when administered. In some embodiments the (human) weekly dosage amount of the compound of formula (I) is about 40 mg to about 200 mg in weeks when administered. In some embodiments the (human) weekly dosage amount of the compound of formula (I) is about 80 mg to about 160 mg in weeks when administered. In some embodiments the (human) weekly dosage amount of the compound of formula (I) is about 90 mg to about 130 mg in weeks when administered.

In some embodiments the (human) weekly dosage amount of the compound of formula (I) is about 140 mg to about 240 mg in weeks when administered. In some embodiments the (human) weekly dosage amount of the compound of formula (I) is about 160 mg to about 220 mg in weeks when administered. In some embodiments the (human) weekly dosage amount of the compound of formula (I) is about 180 mg to about 200 mg in weeks when administered.

Examples of weekly (human) dosage amounts of the compound of formula (I) in weeks when administered include about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, about 190 mg to about 200 mg, about 200 mg to about 210 mg, about 210 mg to about 220 mg, about 220 mg to about 230 mg, about 230 mg to about 240 mg, about 240 mg to about 250 mg, about 250 mg to about 260 mg, about 260 mg to about 270 mg, about 270 mg to about 280 mg, about 280 mg to about 290 mg, about 290 mg to about 300 mg, about 300 mg to about 310 mg, about 310 mg to about 320 mg, about 320 mg to about 330 mg, about 330 mg to about 340 mg, about 340 mg to about 350 mg, about 350 mg to about 360 mg, about 360 mg to about 370 mg, about 370 mg to about 380 mg, about 380 mg to about 390 mg, about 390 mg to about 400 mg, about 400 mg to about 410 mg, about 410 mg to about 420 mg, about 420 mg to about 430 mg, about 430 mg to about 440 mg, about 440 mg to about 450 mg, about 450 mg to about 460 mg, about 460 mg to about 470 mg, and about 470 mg to about 480 mg.

Examples of specific (human) weekly dosage amounts of the compound of formula (I) in weeks when administered include about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, and about 480 mg.

The weekly dose of the compound of formula (I) or a pharmaceutically acceptable salt thereof may be administered in a single administration, e.g. without any pause when administered intravenously. Alternatively the weekly dose may be administered in multiple administrations, e.g. in two or three administrations with pauses in between administrations when administered intravenously, e.g. of at least 30 minutes, e.g. at least an hour, e.g. at least two hours, e.g. at least 4 hours between administrations, e.g. 30 minutes to 12 hours, e.g. 30 minutes to 6 hours between administrations. Such multiple administrations may be on the same day or on separate days, e.g. on consecutive days or e.g. on the third day after the day of initial administration.

The compound of formula (I) or a pharmaceutically acceptable salt thereof is preferably administered intravenously. The duration of the infusion will usually be at least 30 minutes and may be up to 24 hours. In some embodiments the duration of the infusion is 30 minutes to 12 hours, e.g. 30 minutes to 6 hours, e.g. 30 minutes to 3 hours, e.g. one to two hours, e.g. about one hour.

Example (human) weekly dosage amounts of paclitaxel in weeks when administered include about 60 mg/m² to about 90 mg/m², about 60 mg/m² to about 80 mg/m², and about 70 mg/m² to about 80 mg/m².

Examples of specific (human) weekly dosage amounts of paclitaxel in weeks when administered include about 60 mg/m², about 61 mg/m², about 62 mg/m², about 63 mg/m², about 64 mg/m², about 65 mg/m², about 66 mg/m², about 67 mg/m², about 68 mg/m², about 69 mg/m², about 70 mg/m², about 71 mg/m², about 72 mg/m², about 73 mg/m², about 74 mg/m², about 75 mg/m², about 76 mg/m², about 77 mg/m², about 78 mg/m², about 79 mg/m², about 80 mg/m², about 81 mg/m², about 82 mg/m², about 83 mg/m², about 84 mg/m², about 85 mg/m², about 86 mg/m², about 87 mg/m², about 88 mg/m², about 89 mg/m², and about 90 mg/m².

The weekly dose of paclitaxel may be administered in a single administration, e.g. without pause when administered intravenously. Alternatively the weekly dose may be administered in multiple administrations, e.g. in two or three administrations with pauses in between administrations when administered intravenously, e.g. of at least 30 minutes, e.g. at least an hour, e.g. at least two hours, e.g. at least 4 hours between administrations, e.g. 30 minutes to 12 hours, e.g. 30 minutes to 6 hours. Such multiple administrations may be on the same day or on separate days, e.g. on consecutive days or on the third day after the initial administration.

Paclitaxel is preferably administered intravenously. The duration of the infusion will usually be at least 30 minutes and may be up to 24 hours. In some embodiments the duration of the infusion is 30 minutes to 12 hours, e.g. 30 minutes to 6 hours, e.g. 30 minutes to 3 hours, e.g. one to two hours, e.g. about one hour.

In some embodiments the (human) weekly dosage amounts of the compound of formula (I) in weeks when administered and the (human) weekly dosage amount of paclitaxel in weeks when administered is as indicated in any one of embodiments 1A to 21A in Table A.

TABLE A

| Embodiment | Paclitaxel (mg/m²) | Compound of formula (I) (mg) |
|---|---|---|
| 1A | 60-90 | 5-480 |
| 2A | 60-90 | 40-200 |
| 3A | 60-90 | 80-160 |
| 4A | 60-90 | 90-130 |
| 5A | 60-90 | 140-240 |
| 6A | 60-90 | 160-220 |
| 7A | 60-90 | 180-200 |
| 8A | 60-80 | 5-480 |
| 9A | 60-80 | 40-200 |
| 10A | 60-80 | 80-160 |
| 11A | 60-80 | 90-130 |
| 12A | 60-80 | 140-240 |
| 13A | 60-80 | 160-220 |
| 14A | 60-80 | 180-200 |
| 15A | 70-80 | 5-480 |
| 16A | 70-80 | 40-200 |
| 17A | 70-80 | 80-160 |
| 18A | 70-80 | 90-130 |
| 19A | 70-80 | 140-240 |
| 20A | 70-80 | 160-220 |
| 21A | 70-80 | 180-200 |

As per the statement above, embodiment 1A refers to the situation wherein paclitaxel is administered to a patient at a dose of about 60 mg/m² to about 90 mg/m² per week in weeks when administered and the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to embodiments 2A to 21A.

In some embodiments the (human) weekly dosage amounts of the compound of formula (I) in weeks when administered and the (human) weekly dosage amount of paclitaxel in weeks when administered is as indicated in any one of embodiments 1B to 194B in Table B.

TABLE B

| Embodiment | Paclitaxel (mg/m²) | Compound of formula (I) (mg) |
|---|---|---|
| 1B | 60 | 5-10 |
| 2B | 60 | 10-20 |
| 3B | 60 | 20-30 |
| 4B | 60 | 30-40 |
| 5B | 60 | 40-50 |
| 6B | 60 | 50-60 |
| 7B | 60 | 60-70 |
| 8B | 60 | 70-80 |
| 9B | 60 | 80-90 |
| 10B | 60 | 90-100 |
| 11B | 60 | 100-110 |
| 12B | 60 | 110-120 |
| 13B | 60 | 120-130 |
| 14B | 60 | 130-140 |
| 15B | 60 | 140-150 |
| 16B | 60 | 150-160 |
| 17B | 60 | 160-170 |
| 18B | 60 | 170-180 |
| 19B | 60 | 180-190 |
| 20B | 60 | 190-200 |
| 21B | 60 | 200-210 |
| 22B | 60 | 210-220 |
| 23B | 60 | 220-230 |
| 24B | 60 | 230-240 |
| 25B | 60 | 240-250 |
| 26B | 60 | 250-260 |
| 27B | 60 | 260-270 |
| 28B | 60 | 270-280 |
| 29B | 60 | 280-290 |
| 30B | 60 | 290-300 |
| 31B | 60 | 300-310 |
| 32B | 60 | 310-320 |
| 33B | 60 | 320-330 |
| 34B | 60 | 330-340 |
| 35B | 60 | 340-350 |
| 36B | 60 | 350-360 |
| 37B | 60 | 360-370 |
| 38B | 60 | 370-380 |
| 39B | 60 | 380-390 |
| 40B | 60 | 390-400 |
| 41B | 60 | 400-410 |
| 42B | 60 | 410-420 |
| 43B | 60 | 420-430 |
| 44B | 60 | 430-440 |
| 45B | 60 | 440-450 |
| 46B | 60 | 450-460 |
| 47B | 60 | 460-470 |
| 48B | 60 | 470-480 |
| 49B | 60 | 480-490 |
| 50B | 70 | 5-10 |
| 51B | 70 | 10-20 |
| 52B | 70 | 20-30 |
| 53B | 70 | 30-40 |
| 54B | 70 | 40-50 |
| 55B | 70 | 50-60 |
| 56B | 70 | 60-70 |
| 57B | 70 | 70-80 |
| 58B | 70 | 80-90 |
| 59B | 70 | 90-100 |
| 60B | 70 | 100-110 |
| 61B | 70 | 110-120 |
| 62B | 70 | 120-130 |
| 63B | 70 | 130-140 |
| 64B | 70 | 140-150 |
| 65B | 70 | 150-160 |
| 66B | 70 | 160-170 |
| 67B | 70 | 170-180 |
| 68B | 70 | 180-190 |
| 69B | 70 | 190-200 |
| 70B | 70 | 200-210 |
| 71B | 70 | 210-220 |
| 72B | 70 | 220-230 |
| 73B | 70 | 230-240 |
| 74B | 70 | 240-250 |
| 75B | 70 | 250-260 |
| 76B | 70 | 260-270 |
| 77B | 70 | 270-280 |
| 78B | 70 | 280-290 |
| 79B | 70 | 290-300 |
| 80B | 70 | 300-310 |
| 81B | 70 | 310-320 |
| 82B | 70 | 320-330 |
| 83B | 70 | 330-340 |
| 84B | 70 | 340-350 |
| 85B | 70 | 350-360 |
| 86B | 70 | 360-370 |
| 87B | 70 | 370-380 |
| 88B | 70 | 380-390 |
| 89B | 70 | 390-400 |
| 90B | 70 | 400-410 |
| 91B | 70 | 410-420 |
| 92B | 70 | 420-430 |
| 93B | 70 | 430-440 |
| 94B | 70 | 440-450 |
| 95B | 70 | 450-460 |
| 96B | 70 | 460-470 |
| 97B | 70 | 470-480 |
| 98B | 80 | 5-10 |
| 99B | 80 | 10-20 |
| 100B | 80 | 20-30 |
| 101B | 80 | 30-40 |
| 102B | 80 | 40-50 |
| 103B | 80 | 50-60 |
| 104B | 80 | 60-70 |
| 105B | 80 | 70-80 |
| 106B | 80 | 80-90 |
| 107B | 80 | 90-100 |
| 108B | 80 | 100-110 |
| 109B | 80 | 110-120 |
| 110B | 80 | 120-130 |
| 111B | 80 | 130-140 |
| 112B | 80 | 140-150 |
| 113B | 80 | 150-160 |
| 114B | 80 | 160-170 |
| 115B | 80 | 170-180 |
| 116B | 80 | 180-190 |
| 117B | 80 | 190-200 |
| 118B | 80 | 200-210 |
| 119B | 80 | 210-220 |
| 120B | 80 | 220-230 |
| 121B | 80 | 230-240 |
| 122B | 80 | 240-250 |
| 123B | 80 | 250-260 |
| 124B | 80 | 260-270 |
| 125B | 80 | 270-280 |
| 126B | 80 | 280-290 |
| 127B | 80 | 290-300 |
| 128B | 80 | 300-310 |
| 129B | 80 | 310-320 |
| 130B | 80 | 320-330 |
| 131B | 80 | 330-340 |
| 132B | 80 | 340-350 |
| 133B | 80 | 350-360 |
| 134B | 80 | 360-370 |
| 135B | 80 | 370-380 |
| 136B | 80 | 380-390 |
| 137B | 80 | 390-400 |
| 138B | 80 | 400-410 |
| 139B | 80 | 410-420 |
| 140B | 80 | 420-430 |
| 141B | 80 | 430-440 |
| 1428 | 80 | 440-450 |
| 143B | 80 | 450-460 |
| 144B | 80 | 460-470 |
| 145B | 80 | 470-480 |
| 146B | 80 | 480-490 |
| 147B | 90 | 5-10 |
| 148B | 90 | 10-20 |
| 149B | 90 | 20-30 |
| 150B | 90 | 30-40 |
| 151B | 90 | 40-50 |
| 152B | 90 | 50-60 |
| 153B | 90 | 60-70 |
| 154B | 90 | 70-80 |
| 155B | 90 | 80-90 |
| 156B | 90 | 90-100 |
| 157B | 90 | 100-110 |
| 158B | 90 | 110-120 |
| 159B | 90 | 120-130 |
| 160B | 90 | 130-140 |
| 161B | 90 | 140-150 |
| 162B | 90 | 150-160 |
| 163B | 90 | 160-170 |
| 164B | 90 | 170-180 |
| 165B | 90 | 130-190 |
| 166B | 90 | 190-200 |
| 167B | 90 | 200-210 |
| 168B | 90 | 210-220 |
| 169B | 90 | 220-230 |
| 170B | 90 | 230-240 |
| 171B | 90 | 240-250 |
| 172B | 90 | 250-260 |
| 173B | 90 | 260-270 |
| 174B | 90 | 270-280 |
| 175B | 90 | 280-290 |
| 176B | 90 | 290-300 |
| 177B | 90 | 300-310 |
| 178B | 90 | 310-320 |
| 179B | 90 | 320-330 |
| 180B | 90 | 330-340 |
| 181B | 90 | 340-350 |
| 182B | 90 | 350-360 |
| 183B | 90 | 360-370 |
| 184B | 90 | 370-380 |
| 185B | 90 | 380-390 |
| 186B | 90 | 390-400 |
| 187B | 90 | 400-410 |
| 188B | 90 | 410-420 |
| 189B | 90 | 420-430 |
| 190B | 90 | 430-440 |
| 191B | 90 | 440-450 |
| 192B | 90 | 450-460 |
| 193B | 90 | 460-470 |
| 194B | 90 | 470-480 |

As per the statement above, embodiment 1B refers to the situation wherein paclitaxel is administered to a patient at a dose of about 60 mg/m² per week in weeks when administered and the compound of formula (1) or a pharmaceutically acceptable salt thereof is administered to a patient at a dose corresponding to the mole equivalent of about 5 mg to about 10 mg of the free base of the compound of formula (I) per week in weeks when administered. The same applies analogously to embodiments 2B to 194B.

In some embodiments the treatment cycle duration of the compound of formula (I) or a pharmaceutically acceptable salt thereof and of paclitaxel is four weeks and the (human) weekly dosage of the compound of formula (I) and paclitaxel in weeks when administered and the week(s) of administration within the cycle are as indicated in any one of embodiments 1C to 63C in Table C, wherein the compound of formula (I) and paclitaxel are administered according to the same treatment cycle (i.e. starting on the same day).

TABLE C

| | | Paclitaxel | | Compound of formula (I) | |
|---|---|---|---|---|---|
| | Cycle | Weeks | Weekly | Weeks | Weekly |
| Embodi- | duration | of adminis- | dosage | of adminis- | dosage |
| ment | (weeks) | tration | (mm/m²) | tration | (mg) |
| 1C | 4 weeks | 1, 2, 3 | 60-90 | 1 | 5-480 |
| 2C | 4 weeks | 1, 2, 3 | 60-90 | 1 | 40-200 |
| 3C | 4 weeks | 1, 2, 3 | 60-90 | 1 | 80-160 |
| 4C | 4 weeks | 1, 2, 3 | 60-90 | 1 | 90-130 |
| 5C | 4 weeks | 1, 2, 3 | 60-90 | 1 | 140-240 |
| 6C | 4 weeks | 1, 2, 3 | 60-90 | 1 | 160-220 |
| 7C | 4 weeks | 1, 2, 3 | 60-90 | 1 | 180-200 |
| 8C | 4 weeks | 1, 2, 3 | 60-90 | 1, 2 | 5-480 |
| 9C | 4 weeks | 1, 2, 3 | 60-90 | 1, 2 | 40-200 |
| 10C | 4 weeks | 1, 2, 3 | 60-90 | 1, 2 | 80-160 |
| 11C | 4 weeks | 1, 2, 3 | 60-90 | 1, 2 | 90-130 |
| 12C | 4 weeks | 1, 2, 3 | 60-90 | 1, 2 | 140-240 |
| 13C | 4 weeks | 1, 2, 3 | 60-90 | 1, 2 | 160-220 |
| 14C | 4 weeks | 1, 2, 3 | 60-90 | 1, 2 | 180-200 |
| 15C | 4 weeks | 1, 2, 3 | 60-90 | 1, 2, 3 | 5-480 |
| 16C | 4 weeks | 1, 2, 3 | 60-90 | 1, 2, 3 | 40-200 |
| 17C | 4 weeks | 1, 2, 3 | 60-90 | 1, 2, 3 | 80-160 |
| 18C | 4 weeks | 1, 2, 3 | 60-90 | 1, 2, 3 | 90-130 |
| 19C | 4 weeks | 1, 2, 3 | 60-90 | 1, 2, 3 | 140-240 |
| 20C | 4 weeks | 1, 2, 3 | 60-90 | 1, 2, 3 | 160-220 |
| 21C | 4 weeks | 1, 2, 3 | 60-90 | 1, 2, 3 | 180-200 |
| 22C | 4 weeks | 1, 2, 3 | 60-80 | 1 | 5-480 |
| 23C | 4 weeks | 1, 2, 3 | 60-80 | 1 | 40-200 |
| 24C | 4 weeks | 1, 2, 3 | 60-80 | 1 | 80-160 |
| 25C | 4 weeks | 1, 2, 3 | 60-80 | 1 | 90-130 |
| 26C | 4 weeks | 1, 2, 3 | 60-80 | 1 | 140-240 |
| 27C | 4 weeks | 1, 2, 3 | 60-80 | 1 | 160-220 |
| 28C | 4 weeks | 1, 2, 3 | 60-80 | 1 | 180-200 |
| 29C | 4 weeks | 1, 2, 3 | 60-80 | 1, 2 | 5-480 |
| 30C | 4 weeks | 1, 2, 3 | 60-80 | 1, 2 | 40-200 |
| 31C | 4 weeks | 1, 2, 3 | 60-80 | 1, 2 | 80-160 |
| 32C | 4 weeks | 1, 2, 3 | 60-80 | 1, 2 | 90-130 |
| 33C | 4 weeks | 1, 2, 3 | 60-80 | 1, 2 | 140-240 |
| 34C | 4 weeks | 1, 2, 3 | 60-80 | 1, 2 | 160-220 |
| 35C | 4 weeks | 1, 2, 3 | 60-80 | 1, 2 | 180-200 |
| 36C | 4 weeks | 1, 2, 3 | 60-80 | 1, 2, 3 | 5-480 |
| 37C | 4 weeks | 1, 2, 3 | 60-80 | 1, 2, 3 | 40-200 |
| 38C | 4 weeks | 1, 2, 3 | 60-80 | 1, 2, 3 | 80-160 |
| 39C | 4 weeks | 1, 2, 3 | 60-80 | 1, 2, 3 | 90-130 |
| 40C | 4 weeks | 1, 2, 3 | 60-80 | 1, 2, 3 | 140-240 |
| 41C | 4 weeks | 1, 2, 3 | 60-80 | 1, 2, 3 | 160-220 |
| 42C | 4 weeks | 1, 2, 3 | 60-80 | 1, 2, 3 | 180-200 |
| 43C | 4 weeks | 1, 2, 3 | 70-80 | 1 | 5-480 |
| 44C | 4 weeks | 1, 2, 3 | 70-80 | 1 | 40-200 |
| 45C | 4 weeks | 1, 2, 3 | 70-80 | 1 | 80-160 |
| 46C | 4 weeks | 1, 2, 3 | 70-80 | 1 | 90-130 |
| 47C | 4 weeks | 1, 2, 3 | 70-80 | 1 | 140-240 |
| 48C | 4 weeks | 1, 2, 3 | 70-80 | 1 | 160-220 |
| 49C | 4 weeks | 1, 2, 3 | 70-80 | 1 | 180-200 |
| 50C | 4 weeks | 1, 2, 3 | 70-80 | 1, 2 | 5-480 |
| 51C | 4 weeks | 1, 2, 3 | 70-80 | 1, 2 | 40-200 |
| 52C | 4 weeks | 1, 2, 3 | 70-80 | 1, 2 | 80-160 |
| 53C | 4 weeks | 1, 2, 3 | 70-80 | 1, 2 | 90-130 |
| 54C | 4 weeks | 1, 2, 3 | 70-80 | 1, 2 | 140-240 |
| 55C | 4 weeks | 1, 2, 3 | 70-80 | 1, 2 | 160-220 |
| 56C | 4 weeks | 1, 2, 3 | 70-80 | 1, 2 | 180-200 |
| 57C | 4 weeks | 1, 2, 3 | 70-80 | 1, 2, 3 | 5-480 |
| 58C | 4 weeks | 1, 2, 3 | 70-80 | 1, 2, 3 | 40-200 |
| 59C | 4 weeks | 1, 2, 3 | 70-80 | 1, 2, 3 | 80-160 |
| 60C | 4 weeks | 1, 2, 3 | 70-80 | 1, 2, 3 | 90-130 |
| 61C | 4 weeks | 1, 2, 3 | 70-80 | 1, 2, 3 | 140-240 |
| 62C | 4 weeks | 1, 2, 3 | 70-80 | 1, 2, 3 | 160-220 |
| 63C | 4 weeks | 1, 2, 3 | 70-80 | 1, 2, 3 | 180-200 |

As per the statement above, embodiment 1C refers to the situation wherein the compound of formula (I) or a pharmaceutically acceptable salt thereof and paclitaxel are administered (e.g. intravenously) to a patient according to the same four week treatment cycle, wherein the compound of formula (I) or a pharmaceutically acceptable salt thereof is administered in week one of the treatment cycle followed by three rest weeks and paclitaxel is administered in each week during the first three weeks of the treatment cycle followed by a rest week, and wherein paclitaxel is administered to a patient at a dose of about 60 mg/m² to about 90 mg/m² per week in weeks when administered and the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to embodiments 2C to 63C.

In some embodiments the treatment cycle duration of the compound of formula (I) or a pharmaceutically acceptable salt thereof and paclitaxel is 28 days and the (human) weekly dosage of the compound of formula (I) and paclitaxel in weeks when administered and the days of administration within the cycle are as indicated in any one of embodiments 1D to 63D in Table D, wherein the compound of formula (I) and paclitaxel are administered according to the same treatment cycle (i.e. starting on the same day).

TABLE D

| | | Paclitaxel | | Compound of formula (I) | |
|---|---|---|---|---|---|
| | Cycle | Days of | Weekly | Days of | Weekly |
| Embodi- | duration | adminis- | dosage | adminis- | dosage |
| ment | (days) | tration | (mm/m²) | tration | (mg) |
| 1D | 28 | 1, 8, 15 | 60-90 | 1 | 5-480 |
| 2D | 28 | 1, 8, 15 | 60-90 | 1 | 40-200 |
| 3D | 28 | 1, 8, 15 | 60-90 | 1 | 80-160 |
| 4D | 28 | 1, 8, 15 | 60-90 | 1 | 90-130 |
| 5D | 28 | 1, 8, 15 | 60-90 | 1 | 140-240 |
| 6D | 28 | 1, 8, 15 | 60-90 | 1 | 160-220 |
| 7D | 28 | 1, 8, 15 | 60-90 | 1 | 180-200 |
| 8D | 28 | 1, 8, 15 | 60-90 | 1, 8 | 5-480 |
| 9D | 28 | 1, 8, 15 | 60-90 | 1, 8 | 40-200 |
| 10D | 28 | 1, 8, 15 | 60-90 | 1, 8 | 80-160 |
| 11D | 28 | 1, 8, 15 | 60-90 | 1, 8 | 90-130 |
| 12D | 28 | 1, 8, 15 | 60-90 | 1, 8 | 140-240 |
| 13D | 28 | 1, 8, 15 | 60-90 | 1, 8 | 160-220 |
| 14D | 28 | 1, 8, 15 | 60-90 | 1, 8 | 180-200 |
| 15D | 28 | 1, 8, 15 | 60-90 | 1, 8, 15 | 5-480 |
| 16D | 28 | 1, 8, 15 | 60-90 | 1, 8, 15 | 40-200 |
| 17D | 28 | 1, 8, 15 | 60-90 | 1, 8, 15 | 80-160 |
| 18D | 28 | 1, 8, 15 | 60-90 | 1, 8, 15 | 90-130 |
| 19D | 28 | 1, 8, 15 | 60-90 | 1, 8, 15 | 140-240 |
| 20D | 28 | 1, 8, 15 | 60-90 | 1, 8, 15 | 160-220 |
| 21D | 28 | 1, 8, 15 | 60-90 | 1, 8, 15 | 180-200 |
| 22D | 28 | 1, 8, 15 | 60-80 | 1 | 5-480 |
| 23D | 28 | 1, 8, 15 | 60-80 | 1 | 40-200 |
| 24D | 28 | 1, 8, 15 | 60-80 | 1 | 80-160 |
| 25D | 28 | 1, 8, 15 | 60-80 | 1 | 90-130 |
| 26D | 28 | 1, 8, 15 | 60-80 | 1 | 140-240 |
| 27D | 28 | 1, 8, 15 | 60-80 | 1 | 160-220 |
| 28D | 28 | 1, 8, 15 | 60-80 | 1 | 180-200 |
| 29D | 28 | 1, 8, 15 | 60-80 | 1, 8 | 5-480 |
| 30D | 28 | 1, 8, 15 | 60-80 | 1, 8 | 40-200 |
| 31D | 28 | 1, 8, 15 | 60-80 | 1, 8 | 80-160 |
| 32D | 28 | 1, 8, 15 | 60-80 | 1, 8 | 90-130 |
| 33D | 28 | 1, 8, 15 | 60-80 | 1, 8 | 140-240 |
| 34D | 28 | 1, 8, 15 | 60-80 | 1, 8 | 160-220 |
| 35D | 28 | 1, 8, 15 | 60-80 | 1, 8 | 180-200 |
| 36D | 28 | 1, 8, 15 | 60-80 | 1, 8, 15 | 5-480 |
| 37D | 28 | 1, 8, 15 | 60-80 | 1, 8, 15 | 40-200 |
| 38D | 28 | 1, 8, 15 | 60-80 | 1, 8, 15 | 80-160 |
| 39D | 28 | 1, 8, 15 | 60-80 | 1, 8, 15 | 90-130 |
| 40D | 28 | 1, 8, 15 | 60-80 | 1, 8, 15 | 140-240 |
| 41D | 28 | 1, 8, 15 | 60-80 | 1, 8, 15 | 160-220 |
| 42D | 28 | 1, 8, 15 | 60-80 | 1, 8, 15 | 180-200 |
| 43D | 28 | 1, 8, 15 | 70-80 | 1 | 5-480 |
| 44D | 28 | 1, 8, 15 | 70-80 | 1 | 40-200 |
| 45D | 28 | 1, 8, 15 | 70-80 | 1 | 80-160 |
| 46D | 28 | 1, 8, 15 | 70-80 | 1 | 90-130 |
| 47D | 28 | 1, 8, 15 | 70-80 | 1 | 140-240 |
| 48D | 28 | 1, 8, 15 | 70-80 | 1 | 160-220 |
| 49D | 28 | 1, 8, 15 | 70-80 | 1 | 180-200 |
| 50D | 28 | 1, 8, 15 | 70-80 | 1, 8 | 5-480 |
| 51D | 28 | 1, 8, 15 | 70-80 | 1, 8 | 40-200 |
| 52D | 28 | 1, 8, 15 | 70-80 | 1, 8 | 80-160 |
| 53D | 28 | 1, 8, 15 | 70-80 | 1, 8 | 90-130 |
| 54D | 28 | 1, 8, 15 | 70-80 | 1, 8 | 140-240 |
| 55D | 28 | 1, 8, 15 | 70-80 | 1, 8 | 160-220 |
| 56D | 28 | 1, 8, 15 | 70-80 | 1, 8 | 180-200 |
| 57D | 28 | 1, 8, 15 | 70-80 | 1, 8, 15 | 5-480 |
| 58D | 28 | 1, 8, 15 | 70-80 | 1, 8, 15 | 40-200 |
| 59D | 28 | 1, 8, 15 | 70-80 | 1, 8, 15 | 80-160 |
| 60D | 28 | 1, 8, 15 | 70-80 | 1, 8, 15 | 90-130 |
| 61D | 28 | 1, 8, 15 | 70-80 | 1, 8, 15 | 140-240 |
| 62D | 28 | 1, 8, 15 | 70-80 | 1, 8, 15 | 160-220 |
| 63D | 28 | 1, 8, 15 | 70-80 | 1, 8, 15 | 180-200 |

As per the statement above, embodiment 1D refers to the situation wherein the compound of formula (I) or a pharmaceutically acceptable salt thereof and paclitaxel are administered (e.g. intravenously) to a patient according to the same 28 day treatment cycle, wherein the compound of formula (I) or a pharmaceutically acceptable salt thereof is administered on day 1 of the treatment cycle and paclitaxel is administered on days 1, 8 and 15 of the treatment cycle, and wherein paclitaxel is administered to a patient at a dose of about 60 mg/m² to about 90 mg/m² per week in weeks when administered and the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to embodiments 2D to 63D.

In some embodiments, the mass ratio of the weekly dose of paclitaxel (in mg/m²) to the weekly dose of the compound of formula (I) (in mg) in weeks when administered is e.g. about 0.1:1 to about 18.0:1, e.g. about 0.1:1 to about 16.0:1, e.g. about 0.3:1 to about 2.3:1, e.g. about 0.3:1 to about 2.0:1, e.g. about 0.4:1 to about 2.0:1, e.g. about 0.4:1 to about 1.1:1, e.g. about 0.4:1 to about 1.0:1, e.g. about 0.5:1 to about 1.0:1, e.g. about 0.5:1 to about 0.9:1, e.g. about 0.3:1 to about 0.6:1, e.g. about 0.3:1 to about 0.5:1, e.g. about 0.4:1 to about 0.5:1, e.g. about 0.3:1 to about 0.4:1. In some embodiments, the mass ratio of the weekly dose of paclitaxel (in mg/m²) to the weekly dose of the compound of formula (I) (in mg) in weeks when administered is about 0.4:1 to about 2.0:1.

In some embodiments, the mass ratio of the weekly dose of paclitaxel (in mg/m²) to the weekly dose of the compound of formula (I) (in mg) in weeks when administered is e.g. up to about 18.0:1, e.g. up to about 16.0:1, e.g. up to about 2.3:1, e.g. up to about 2.0:1, e.g. up to about 1.1:1, e.g. up to about 1.0:1, e.g. up to about 0.9:1, e.g. up to about 0.6:1, e.g. up to about 0.5:1, e.g. up to about 0.4:1. In some embodiments, the mass ratio of the weekly dose of paclitaxel (in mg/m²) to the weekly dose of the compound of formula (I) (in mg) in weeks when administered is up to about 2.0:1.

In some embodiments, the mass ratio of the weekly dose of paclitaxel (in mg/m²) to the weekly dose of the compound of formula (I) (in mg) in weeks when administered is e.g. at least about 0.1:1, e.g. at least about 0.3:1, e.g. at least about 0.4:1, e.g. at least about 0.5:1. In some embodiments, the mass ratio of the weekly dose of paclitaxel (in mg/m²) to the weekly dose of the compound of formula (I) (in mg) in weeks when administered is at least about 0.4:1.

In some embodiments when the compound of formula (I) or a pharmaceutically acceptable salt thereof and the taxane anticancer drug (e.g. paclitaxel) are administered on the same day the compound of formula (I) or a pharmaceutically acceptable salt thereof is administered before the taxane anticancer drug (e.g. paclitaxel). In some embodiments when the compound of formula (I) or a pharmaceutically acceptable salt thereof and the taxane anticancer drug (e.g. paclitaxel) are administered on the same day the compound of formula (I) or a pharmaceutically acceptable salt thereof is administered after the taxane anticancer drug (e.g. paclitaxel). Usually there will be a pause between administration of the compound of formula (I) or a pharmaceutically acceptable salt thereof and the taxane anticancer drug (e.g. paclitaxel), e.g. of at least 30 minutes, e.g. at least an hour, e.g. at least two hours, e.g. at least 4 hours, e.g. up to 24 hours, e.g. 30 minutes to 24 hours, e.g. 30 minutes to 12 hours, e.g. 30 minutes to 6 hours between administrations.

In some embodiments the taxane anticancer drug (e.g. paclitaxel) is administered to the subject before the compound of formula (I) or a pharmaceutically acceptable salt thereof is administered to the subject. In some embodiments when the taxane anticancer drug (e.g. paclitaxel) and the compound of formula (I) or a pharmaceutically acceptable salt thereof are both administered to the subject within a 48 hour period the taxane anticancer drug (e.g. paclitaxel) is administered to the subject before the compound of formula (I) or a pharmaceutically acceptable salt thereof is administered to the subject. In some embodiments when the taxane anticancer drug (e.g. paclitaxel) and the compound of formula (I) or a pharmaceutically acceptable salt thereof are both administered to the subject within a 24 hour period then the taxane anticancer drug (e.g. paclitaxel) is administered to the subject before the compound of formula (I) or a pharmaceutically acceptable salt thereof is administered to the subject.

In some embodiments the taxane anticancer drug (e.g. paclitaxel) is administered to the subject at least about 30 minutes before the compound of formula (I) or pharmaceutically acceptable salt thereof is administered to the subject. In some embodiments the taxane anticancer drug (e.g. paclitaxel) is administered to the subject at least about 1 hour before the compound of formula (I) or pharmaceutically acceptable salt thereof is administered to the subject. In some embodiments the taxane anticancer drug (e.g. paclitaxel) is administered to the subject at least about 4 hours before the compound of formula (I) or pharmaceutically acceptable salt thereof is administered to the subject. In some embodiments the taxane anticancer drug (e.g. paclitaxel) is administered to the subject at least about 24 hours before the compound of formula (I) or pharmaceutically acceptable salt thereof is administered to the subject.

In some embodiments when the taxane anticancer drug (e.g. paclitaxel) and the compound of formula (I) or a pharmaceutically acceptable salt thereof are both administered to the subject within a 24 hour period then the taxane anticancer drug (e.g. paclitaxel) is administered to the subject at least about 30 minutes before the compound of formula (I) or a pharmaceutically acceptable salt thereof is administered to the subject. In some embodiments when the taxane anticancer drug (e.g. paclitaxel) and the compound of formula (I) or a pharmaceutically acceptable salt thereof are both administered to the subject within a 24 hour period then the taxane anticancer drug (e.g. paclitaxel) is administered to the subject at least about 1 hour before (the scheduled dose of) the compound of formula (I) or a pharmaceutically acceptable salt thereof is administered to the subject. In some embodiments when the taxane anticancer drug (e.g. paclitaxel) and the compound of formula (I) or a pharmaceutically acceptable salt thereof are both administered to the subject within a 24 hour period the taxane anticancer drug (e.g. paclitaxel) is administered to the subject at least 4 hours before the compound of formula (I) or a pharmaceutically acceptable salt thereof is administered to the subject. In some embodiments when the taxane anticancer drug (e.g. paclitaxel) and the compound of formula (I) or a pharmaceutically acceptable salt thereof are both administered to the subject within a 48 hour period then the taxane anticancer drug (e.g. paclitaxel) is administered to the subject at least about 24 hours before the compound of formula (I) or a pharmaceutically acceptable salt thereof is administered to the subject.

In some embodiments when the taxane anticancer drug (e.g. paclitaxel) and the compound of formula (I) or a pharmaceutically acceptable salt thereof are both administered to the subject within a 48 hour period the taxane anticancer drug (e.g. paclitaxel) is administered to the subject about 30 minutes to about 24 hours before the compound of formula (I) or a pharmaceutically acceptable salt thereof is administered to the subject. In some embodiments when the taxane anticancer drug (e.g. paclitaxel) and the compound of formula (I) or a pharmaceutically acceptable salt thereof are both administered to the subject within a 24 hour period then the taxane anticancer drug (e.g. paclitaxel) is administered to the subject about 30 minutes to about 12 hours before the compound of formula (I) or a pharmaceutically acceptable salt thereof is administered to the subject. In some embodiments when the taxane anticancer drug (e.g. paclitaxel) and the compound of formula (I) or a pharmaceutically acceptable salt thereof are both administered to the subject within a 24 hour period then the taxane anticancer drug (e.g. paclitaxel) is administered to the subject about 30 minutes to about 6 hours before the compound of formula (I) or a pharmaceutically acceptable salt thereof is administered to the subject. In some embodiments when the taxane anticancer drug (e.g. paclitaxel) and the compound of formula (I) or a pharmaceutically acceptable salt thereof are both administered to the subject within a 24 hour period then the taxane anticancer drug (e.g. paclitaxel) is administered to the subject about 2 to about 4 hours before the compound of formula (I) or a pharmaceutically acceptable salt thereof is administered to the subject. In some embodiments when the taxane anticancer drug (e.g. paclitaxel) and the compound of formula (I) or a pharmaceutically acceptable salt thereof are both administered to the subject within a 24 hour period then the taxane anticancer drug (e.g. paclitaxel) is administered to the subject about 4 to about 6 hours before the compound of formula (I) or a pharmaceutically acceptable salt thereof is administered to the subject.

The potential for administration of the taxane anticancer drug (e.g. paclitaxel) prior to the compound of formula (I) or a pharmaceutically acceptable salt thereof as described above applies in particular to the embodiments described in Tables A, B, C and D.

Generally, the compound of formula (I) or a pharmaceutically acceptable salt thereof and paclitaxel will be administered at dosages that do not exceed the maximum tolerated dose (MTD) for a particular mode of administration and indication, as determined in a clinical dose escalation study.

### Formulations

The compound of formula (I) or a pharmaceutically acceptable salt thereof and the taxane anticancer drug or a pharmaceutically acceptable salt thereof (e.g. paclitaxel) may be provided as a combined medicament, but each will usually be provided as a single-agent medicaments. Separate pharmaceutical compositions have a number of advantages, for example, to allow different dosing schedules, different dosages and/or different routes of administration for each compound. When provided as single-agent medicaments the combination may be for separate, simultaneous or sequential administration.

The compounds of the invention may be formulated as pharmaceutical compositions for non-parenteral administration, such as nasal, buccal, rectal, pulmonary, vaginal, sublingual, topical, transdermal, ophthalmic, otic or, especially, for oral administration, e.g. in the form of oral solid dosage forms, e.g. granules, pellets, powders, tablets, film or sugar coated tablets, effervescent tablets, hard and soft gelatin or HPMC capsules, coated as applicable, orally disintegrating tablets, oral solutions, lipid emulsions or suspensions, or for parenteral administration, such as intravenous, intramuscular, or subcutaneous, intrathecal, intradermal or epidural administration, to mammals, especially humans, e.g. in the form of solutions, lipid emulsions or suspensions containing microparticles or nanoparticles. Liposomal formulations provide a convenient way of co-formulating compounds with different solubility (see e.g. Bulbake et al. Pharmaceutics 2017, 9(2):12). The compositions may comprise the active ingredient(s) alone or, preferably, together with a pharmaceutically acceptable carrier.

The pharmaceutical compositions can be processed with pharmaceutically inert, inorganic or organic excipients for the production of oral solid dosage forms, e.g. granules, pellets, powders, tablets, film or sugar coated tablets, effervescent tablets, hard gelatin or HPMC capsules or orally disintegrating tablets. Fillers e.g. lactose, cellulose, mannitol, sorbitol, calcium phosphate, starch or derivatives thereof, binders e.g. cellulose, starch, polyvinylpyrrolidone, or derivatives thereof, glidants e.g. talcum, stearic acid or its salts, flowing agents e.g. fumed silica, can be used as such excipients for formulating and manufacturing of oral solid dosage forms, such as granules, pellets, powders, tablets, film or sugar coated tablets, effervescent tablets, hard gelatin or HPMC capsules, or orally disintegrating tablets. Suitable excipients for soft gelatin capsules are e.g. vegetable oils, waxes, fats, semisolid and liquid polyols etc.

Suitable excipients for the manufacture of oral solutions, lipid emulsions or suspensions are e.g. water, alcohols, polyols, saccharose, invert sugar, glucose etc. Suitable excipients for parenteral formulations are e.g. water, alcohols, polyols, glycerol, vegetable oils, lecithin, surfactants etc. Moreover, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain other therapeutically valuable substances.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor® EL or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. For intravenous injection of strongly lipophilic molecules it can be advantageous to include solubilizers in the formulation, for example surfactants, polymeric surfactants, polymers, complexing agents and/or co-solvents, which may significantly increase the solubility of the compounds in water. Examples of solubilizers include polyethylene glycol, propylene glycol, ethanol, glycerol and cyclodextrins (e.g. sulfobutyl ether-β-cyclodextrins).

In some embodiments the compound of formula (I) as the free base is provided as a pharmaceutical composition comprising a β-cyclodextrin e.g. for intravenous administration. The β-cyclodextrin may be sulfobutyl ether-β-cyclodextrin, e.g. CAS 182410-00-0, such as Captisol™ (Ligand) or Dexolve™ (Cyclolab).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In addition pharmaceutical compositions used in the invention optionally include buffers such as phosphate, citrate, or other organic acids; antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, or other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, the pharmaceutical compositions contain a pharmaceutically acceptable preservative. In some embodiments the preservative concentration ranges from 0.1 to 2.0 percent, typically v/v. Suitable preservatives include those known in the pharmaceutical arts, such as benzyl alcohol, phenol, m-cresol, methylparaben, and propylparaben.

In some embodiments the compound of formula (I) or a pharmaceutically acceptable salt thereof is formulated for intravenous administration with a suitable acceptable carrier. In some embodiments the taxane anticancer drug or a pharmaceutically acceptable salt thereof (e.g. paclitaxel) is formulated for intravenous administration with a suitable acceptable carrier.

The paclitaxel formulation sold under the brand name Taxol® contains Cremophor® EL (polyethoxylated castor oil) and ethanol which is diluted with a suitable parenteral fluid for intravenous administration. Alternative paclitaxel formulations include those such as:
Abraxane®: a nanoparticle formulation of paclitaxel and human serum albumin
Taxoprexin®: a prodrug of paclitaxel chemically bound to the fatty acid, docosahexaenoic acid
Paclical® poliglumex: paclitaxel conjugated to poly-(l-glutamic acid)
ANG1005: Paclitaxel linked to angiopep-2 (brain peptide vector)
Paccal®: a formulation using an excipient composed of a surfactant-based derivative of retinoic acid (XR-17) that results in a nanoparticle micellar preparation with high water solubility that eliminates the need for Cremophor®
(Khanna et al. J Vet Intern Med, 2015; 29 (4): 1006-1012).

### Kits

The invention also provides pharmaceutical products such as kits which may include a container with the compound of formula (I) or a pharmaceutically acceptable salt thereof and the taxane anticancer drug or a pharmaceutically acceptable salt thereof (e.g. paclitaxel), which can be provided in amounts sufficient for administration, e.g. in pharmaceutically acceptable amounts. The kits can thus include multiple containers that each include pharmaceutically effective amounts of the active ingredients. Optionally, instruments and/or devices necessary for administering the pharmaceutical composition(s) can also be included in the kits. Furthermore, the kits can include additional components, such as instructions or administration schedules, for treating a patient with a neoplastic disease with the combinations of the invention.

Accordingly, in a further aspect the invention provides a pharmaceutical product such as a kit for use in treating a neoplastic disease e.g. as described herein, wherein the compound of formula (I) or a pharmaceutically acceptable salt thereof and the taxane anticancer drug or a pharmaceutically acceptable salt thereof (e.g. paclitaxel) are provided as single agent medicaments. In some embodiments, the kit further comprises instructions for simultaneous, separate or sequential administration thereof for use in the treatment of a neoplastic disease.

The following numbered paragraphs describe particular embodiments of the invention.

Paragraph 1. A pharmaceutical combination comprising (a) a compound of formula (I) or a pharmaceutically acceptable salt thereof; and (b) a taxane anticancer drug or a pharmaceutically acceptable salt thereof.

Paragraph 2. The pharmaceutical combination according to Paragraph 1, wherein the taxane anticancer drug or a pharmaceutically acceptable salt thereof is selected from paclitaxel, docetaxel, cabazitaxel, larotaxel, milataxel, ortataxel and tesetaxel, including pharmaceutically acceptable salts thereof.

Paragraph 3. The pharmaceutical combination according to Paragraph 1, wherein the taxane anticancer drug or a pharmaceutically acceptable salt thereof is paclitaxel.

Paragraph 4. The pharmaceutical combination according to any one of Paragraphs 1 to 3, wherein the compound of formula I or a pharmaceutically acceptable derivative thereof and the taxane anticancer drug or a pharmaceutically acceptable salt thereof are comprised in separate formulations, each formulation preferably for intravenous administration.

Paragraph 5. The pharmaceutical combination according to any one of Paragraphs 1 to 4, wherein the molar ratio of the taxane anticancer drug or a pharmaceutically acceptable salt thereof (e.g. paclitaxel) to the compound of formula (I) or a pharmaceutically acceptable salt thereof per weekly dose is about 0.1:1 to about 18.8:1.

Paragraph 6. The pharmaceutical combination according to any one of Paragraphs 1 to 4, wherein the molar ratio of the taxane anticancer drug or a pharmaceutically acceptable salt thereof (e.g. paclitaxel) to the compound of formula (I) or a pharmaceutically acceptable salt thereof per weekly dose is e.g. about 0.3:1 to about 2.3:1.

Paragraph 7. The pharmaceutical combination according to any one of Paragraphs 1 to 4, wherein the molar ratio of the taxane anticancer drug or a pharmaceutically acceptable salt thereof (e.g. paclitaxel) to the compound of formula (I) or a pharmaceutically acceptable salt thereof per weekly dose is e.g. about 0.4:1 to about 2.1:1.

Paragraph 8. The pharmaceutical combination according to any one of Paragraphs 1 to 4, wherein the molar ratio of the taxane anticancer drug or a pharmaceutically acceptable salt thereof (e.g. paclitaxel) to the compound of formula (I) or a pharmaceutically acceptable salt thereof per weekly dose is e.g. about 0.6:1 to about 0.9:1.

Paragraph 9. A method for treating a neoplastic disease in a subject in need thereof, in particular a human, comprising administering to the subject a therapeutically effective amount of a pharmaceutical combination as defined in any one of Paragraphs 1 to 8.

Paragraph 10. The method according to Paragraph 9, wherein the pharmaceutical combination is a compound of formula (I) or a pharmaceutically acceptable salt thereof and paclitaxel.

Paragraph 11. The method according to Paragraph 9 or Paragraph 10, wherein the compound of formula (I) or a pharmaceutically acceptable derivative thereof and the taxane anticancer drug or a pharmaceutically acceptable salt thereof (e.g. paclitaxel) are administered intravenously to the subject.

Paragraph 12. The method according to any one of Paragraphs 9 to 11, wherein the compound of formula (I) or a pharmaceutically acceptable salt thereof and the taxane anticancer drug or a pharmaceutically acceptable salt thereof (e.g. paclitaxel) are administered to the subject according to a four week treatment cycle (preferably the same four week treatment cycle), wherein the compound of formula (I) or a pharmaceutically acceptable salt thereof is administered in week one of the treatment cycle followed by three rest weeks and the taxane anticancer drug or a pharmaceutically acceptable salt thereof (e.g. paclitaxel) is administered in each week during the first three weeks of the treatment cycle followed by a rest week.

Paragraph 13. The method according to Paragraph 12, wherein the compound of formula (I) or a pharmaceutically acceptable salt thereof is administered on day 1 of the treatment cycle and the taxane anticancer drug or a pharmaceutically acceptable salt thereof (e.g. paclitaxel) is administered on days 1, 8 and 15 of the treatment cycle.

Paragraph 14. The method according to any one of Paragraphs 9 to 11, wherein the compound of formula (I) or a pharmaceutically acceptable salt thereof and the taxane anticancer drug or a pharmaceutically acceptable salt thereof (e.g. paclitaxel) are administered to the subject according to a four week treatment cycle (preferably the same four week treatment cycle), wherein the compound of formula (I) or a pharmaceutically acceptable salt thereof is administered in week one and in week three of the treatment cycle, with weeks two and four being rest weeks, and the taxane anticancer drug or a pharmaceutically acceptable salt thereof (e.g. paclitaxel) is administered in each week during the first three weeks of the treatment cycle followed by a rest week.

Paragraph 15. The method according to Paragraph 14, wherein the compound of formula (I) or a pharmaceutically acceptable salt thereof is administered on days 1 and 15 of the treatment cycle and the taxane anticancer drug or a pharmaceutically acceptable salt thereof (e.g. paclitaxel) is administered on days 1, 8 and 15 of the treatment cycle.

Paragraph 16. The method according to any one of Paragraphs 9 to 11, wherein the compound of formula (I) or a pharmaceutically acceptable salt thereof and the taxane anticancer drug or a pharmaceutically acceptable salt thereof (e.g. paclitaxel) are administered to the subject according to a four week treatment cycle (preferably the same four week treatment cycle), wherein the compound of formula (I) or a pharmaceutically acceptable salt thereof is administered in each week during the first three weeks of the treatment cycle followed by a rest week, and the taxane anticancer drug or a pharmaceutically acceptable salt thereof (e.g. paclitaxel) is administered in each week during the first three weeks of the treatment cycle followed by a rest week.

Paragraph 17. The method according to Paragraph 16, wherein the compound of formula (I) or a pharmaceutically acceptable salt thereof is administered on days 1, 8 and 15 of the treatment cycle and the taxane anticancer drug or a pharmaceutically acceptable salt thereof (e.g. paclitaxel) is administered on days 1, 8 and 15 of the treatment cycle.

Paragraph 18. The method according to any one of Paragraphs 9 to 17, wherein the molar ratio of the taxane anticancer drug or a pharmaceutically acceptable salt thereof (e.g. paclitaxel) to the compound of formula (I) or a pharmaceutically acceptable salt thereof per weekly dose is about 0.1:1 to about 18.8:1.

Paragraph 19. The method according to any one of Paragraphs 9 to 17, wherein the molar ratio of the taxane anticancer drug or a pharmaceutically acceptable salt thereof (e.g. paclitaxel) to the compound of formula (I) or a pharmaceutically acceptable salt thereof per weekly dose is e.g. about 0.3:1 to about 2.3:1.

Paragraph 20. The method according to any one of Paragraphs 9 to 17, wherein the molar ratio of the taxane anticancer drug or a pharmaceutically acceptable salt thereof (e.g. paclitaxel) to the compound of formula (I) or a pharmaceutically acceptable salt thereof per weekly dose is e.g. about 0.4:1 to about 2.1:1.

Paragraph 21. The method according to any one of Paragraphs 9 to 17, wherein the molar ratio of the taxane anticancer drug or a pharmaceutically acceptable salt thereof (e.g. paclitaxel) to the compound of formula (I) or a pharmaceutically acceptable salt thereof per weekly dose is e.g. about 0.6:1 to about 0.9:1.

Paragraph 22. The method according to any one of Paragraphs 9 to 17, wherein the compound of formula (I) or a pharmaceutically acceptable salt thereof is administered to a patient at a dose corresponding to the mole equivalent of about 5 mg to about 480 mg of the free base of the compound of formula (I) per week in weeks when administered.

Paragraph 23. The method according to any one of Paragraphs 9 to 17, wherein the compound of formula (I) or a pharmaceutically acceptable salt thereof is administered to a patient at a dose corresponding to the mole equivalent of about 40 mg to about 200 mg of the free base of the compound of formula (I) per week in weeks when administered.

Paragraph 24. The method according to any one of Paragraphs 9 to 17, wherein the compound of formula (I) or a pharmaceutically acceptable salt thereof is administered to a patient at a dose corresponding to the mole equivalent of about 80 mg to about 160 mg of the free base of the compound of formula (I) per week in weeks when administered.

Paragraph 25. The method according to any one of Paragraphs 9 to 21, wherein the compound of formula (I) or a pharmaceutically acceptable salt thereof is administered to a patient at a dose corresponding to the mole equivalent of about 90 mg to about 130 mg of the free base of the compound of formula (I) per week in weeks when administered.

Paragraph 26. The method according to any one of paragraphs 9 to 21, wherein the compound of formula (I) or a pharmaceutically acceptable salt thereof is administered to a patient at a dose corresponding to the mole equivalent of about 140 mg to about 240 mg of the free base of the compound of formula (I) per week in weeks when administered.

Paragraph 27. The method according to any one of paragraphs 9 to 21, wherein the compound of formula (I) or a pharmaceutically acceptable salt thereof is administered to a patient at a dose corresponding to the mole equivalent of about 160 mg to about 220 mg of the free base of the compound of formula (I) per week in weeks when administered.

Paragraph 28. The method according to any one of paragraphs 9 to 21, wherein the compound of formula (I) or a pharmaceutically acceptable salt thereof is administered to a patient at a dose corresponding to the mole equivalent of about 180 mg to about 200 mg of the free base of the compound of formula (I) per week in weeks when administered.

Paragraph 29. The method according to any one of Paragraphs 22 to 28, wherein the taxane anticancer drug or a pharmaceutically acceptable salt thereof is paclitaxel and paclitaxel is administered to a patient at a dose of about 60 mg/m² to about 90 mg/m² per week in weeks when administered.

Paragraph 30. The method according to any one of Paragraphs 22 to 28, wherein the taxane anticancer drug or a pharmaceutically acceptable salt thereof is paclitaxel and paclitaxel is administered to a patient at a dose of about 60 mg/m² to about 80 mg/m² per week in weeks when administered.

Paragraph 31. The method according to any one of Paragraphs 22 to 28, wherein the taxane anticancer drug or a pharmaceutically acceptable salt thereof is paclitaxel and paclitaxel is administered to a patient at a dose of about 70 mg/m² to about 80 mg/m² per week in weeks when administered.

Paragraph 32. The method according to any one of Paragraphs 9 to 31, wherein the compound of formula (I) or a pharmaceutically acceptable salt thereof and the taxane anticancer drug or a pharmaceutically acceptable salt thereof (e.g. paclitaxel) are administered on the same day and wherein the compound of formula (I) or a pharmaceutically acceptable salt thereof is administered to the subject before the taxane anticancer drug or a pharmaceutically acceptable salt thereof (e.g. paclitaxel).

Paragraph 33. The method according to any one of Paragraphs 9 to 31, wherein the compound of formula (I) or a pharmaceutically acceptable salt thereof and the taxane anticancer drug or a pharmaceutically acceptable salt thereof (e.g. paclitaxel) are administered on the same day and wherein the compound of formula (I) or a pharmaceutically acceptable salt thereof is administered to the subject after the taxane anticancer drug or a pharmaceutically acceptable salt thereof (e.g. paclitaxel).

Paragraph 34. The method according to Paragraph 32 or Paragraph 33, wherein the time interval between the administrations is at least about 4 hours.

Paragraph 35. The method according to Paragraph 32 or Paragraph 33, wherein the time interval between the administrations is at least about 2 hours.

Paragraph 36. The method according to any one of Paragraphs 9 to 35, wherein the neoplastic disease is a solid tumor.

Paragraph 37. The method according to any one of Paragraphs 9 to 36, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, miscellaneous myeloproliferative disorders, lymphoproliferative disorders and myelodysplastic syndromes.

Paragraph 38. The method according to any one of Paragraphs 9 to 37, wherein the neoplastic disease is one which is treatable by inhibition of PLK1 in addition to a treatment with a taxane anticancer drug (e.g. paclitaxel) and a TTK inhibitor (e.g. the compound of formula (I)).

Paragraph 39. The method according to any one of Paragraphs 9 to 38, wherein the neoplastic disease is cancer.

Paragraph 40. The method according to Paragraph 39, wherein the cancer in terms of the organs and parts of the body affected is selected from brain, breast (including triple negative breast cancer and luminal B breast cancer), cervix, ovaries, colon, rectum (including colon and rectum i.e. colorectal cancer), lung (including small cell lung cancer, non-small cell lung cancer, large cell lung cancer and mesothelioma), endocrine system, bone, adrenal gland, thymus, liver, stomach, intestine (including gastric cancer), pancreas, bone marrow, hematological malignancies (such as lymphoma, leukemia, myeloma or lymphoid malignancies), bile duct, bladder, urinary tract, kidneys, skin, thyroid, head, neck, prostate and testis.

Paragraph 41. The method according to Paragraph 39, wherein the cancer is selected from breast cancer (including triple negative breast cancer and luminal B breast cancer), gastric cancer, colorectal cancer, liver cancer (including hepatocellular cancer), endometrial cancer, ovarian cancer, esophageal cancer, lung cancer (including non-small cell lung cancer), Kaposi's sarcoma, cervical cancer, pancreatic cancer, melanoma, prostate cancer, bladder cancer and leukemia, e.g. acute myeloid leukemia (AML) (including Complex Karyotype AML).

Paragraph 42. The method according to Paragraph 39, wherein the cancer is breast cancer.

Paragraph 43. The method according to Paragraph 39, wherein the cancer is triple negative breast cancer.

Paragraph 44. The method according to Paragraph 39, wherein the cancer is luminal B breast cancer.

Paragraph 45. A method for treating a neoplastic disease in a subject in need thereof comprising administering to the subject a therapeutically effective amount of the compound of formula (I) or a pharmaceutically acceptable salt thereof as defined in Paragraph 1, wherein said subject is undergoing or will undergo treatment with a taxane anticancer drug or a pharmaceutically acceptable salt thereof as defined in any one of Paragraphs 1 to 3 (e.g. paclitaxel).

Paragraph 46. The method according to Paragraph 45, wherein the compound of formula (I) or a pharmaceutically acceptable salt thereof is administered to the subject as defined in any one of Paragraphs 11 to 35.

Paragraph 47. The method according to Paragraph 40 or Paragraph 41, wherein the neoplastic disease is as defined in any one of Paragraphs 36 to 44.

Paragraph 48. A method for treating a neoplastic disease in a subject in need thereof comprising administering to the subject a therapeutically effective amount of a taxane anticancer drug or a pharmaceutically acceptable salt thereof as defined in any one of Paragraphs 1 to 3 (e.g. paclitaxel), wherein said subject is undergoing or will undergo treatment with the compound of formula (I) or a pharmaceutically acceptable salt thereof as defined in Paragraph 1.

Paragraph 49. The method according to Paragraph 48, wherein the taxane anticancer drug or a pharmaceutically acceptable salt thereof (e.g. paclitaxel) is administered to the subject as defined in any one of Paragraphs 11 to 35.

Paragraph 50. The method according to Paragraph 48 or Paragraph 49, wherein the neoplastic disease is as defined in any one of Paragraphs 36 to 44.

Paragraph 51. A pharmaceutical combination as defined in any one of Paragraphs 1 to 8, for use in the treatment of a neoplastic disease in a subject, in particular a human.

Paragraph 52. The pharmaceutical combination for use according to paragraph 51, wherein the pharmaceutical combination is a compound of formula (I) or a pharmaceutically acceptable salt thereof and paclitaxel.

Paragraph 53. The pharmaceutical combination for use according to Paragraph 51 or Paragraph 52, wherein the compound of formula (I) or a pharmaceutically acceptable derivative thereof and the taxane anticancer drug or a pharmaceutically acceptable salt thereof are administered intravenously to the subject.

Paragraph 54. The pharmaceutical combination for use according to any one of Paragraphs 51 to 53, wherein the compound of formula (I) or a pharmaceutically acceptable salt thereof and the taxane anticancer drug or a pharmaceutically acceptable salt thereof (e.g. paclitaxel) are administered to the subject according to a four week treatment cycle (preferably the same four week treatment cycle), wherein the compound of formula (I) or a pharmaceutically acceptable salt thereof is administered in week one of the treatment cycle followed by three rest weeks and the taxane anticancer drug or a pharmaceutically acceptable salt thereof (e.g. paclitaxel) is administered in each week during the first three weeks of the treatment cycle followed by a rest week.

Paragraph 55. The pharmaceutical combination for use according to Paragraph 54, wherein the compound of formula (I) or a pharmaceutically acceptable salt thereof is administered on day 1 of the treatment cycle and the taxane anticancer drug or a pharmaceutically acceptable salt thereof (e.g. paclitaxel) is administered on days 1, 8 and 15 of the treatment cycle.

Paragraph 56. The pharmaceutical combination for use according to any one of Paragraphs 51 to 53, wherein the compound of formula (I) or a pharmaceutically acceptable salt thereof and the taxane anticancer drug or a pharmaceutically acceptable salt thereof (e.g. paclitaxel) are administered to the subject according to a four week treatment cycle (preferably the same four week treatment cycle), wherein the compound of formula (I) or a pharmaceutically acceptable salt thereof is administered in week one and in week three of the treatment cycle, with weeks two and four being rest weeks, and the taxane anticancer drug or a pharmaceutically acceptable salt thereof (e.g. paclitaxel) is administered in each week during the first three weeks of the treatment cycle followed by a rest week.

Paragraph 57. The pharmaceutical combination for use according to Paragraph 56, wherein the compound of formula (I) or a pharmaceutically acceptable salt thereof is administered on days 1 and 15 of the treatment cycle and the taxane anticancer drug or a pharmaceutically acceptable salt thereof (e.g. paclitaxel) is administered on days 1, 8 and 15 of the treatment cycle.

Paragraph 58. The pharmaceutical combination for use according to any one of Paragraphs 51 to 53, wherein the compound of formula (I) or a pharmaceutically acceptable salt thereof and the taxane anticancer drug or a pharmaceutically acceptable salt thereof (e.g. paclitaxel) are administered to the subject according to a four week treatment cycle (preferably the same four week treatment cycle), wherein the compound of formula (I) or a pharmaceutically acceptable salt thereof is administered in each week during the first three weeks of the treatment cycle followed by a rest week, and the taxane anticancer drug or a pharmaceutically acceptable salt thereof (e.g. paclitaxel) is administered in each week during the first three weeks of the treatment cycle followed by a rest week.

Paragraph 59. The pharmaceutical combination for use according to Paragraph 58, wherein the compound of formula (I) or a pharmaceutically acceptable salt thereof is administered on days 1, 8 and 15 of the treatment cycle and the taxane anticancer drug or a pharmaceutically acceptable salt thereof (e.g. paclitaxel) is administered on days 1, 8 and 15 of the treatment cycle.

Paragraph 60. The pharmaceutical combination for use according to any one of Paragraphs 51 to 59, wherein the molar ratio of the taxane anticancer drug or a pharmaceutically acceptable salt thereof (e.g. paclitaxel) to the compound of formula (I) or a pharmaceutically acceptable salt thereof per weekly dose is about 0.1:1 to about 18.8:1.

Paragraph 61. The pharmaceutical combination for use according to any one of Paragraphs 51 to 59, wherein the molar ratio of the taxane anticancer drug or a pharmaceutically acceptable salt thereof (e.g. paclitaxel) to the compound of formula (I) or a pharmaceutically acceptable salt thereof per weekly dose is e.g. about 0.3:1 to about 2.3:1.

Paragraph 62. The pharmaceutical combination for use according to any one of Paragraphs 51 to 59, wherein the molar ratio of the taxane anticancer drug or a pharmaceutically acceptable salt thereof (e.g. paclitaxel) to the compound of formula (I) or a pharmaceutically acceptable salt thereof per weekly dose is e.g. about 0.4:1 to about 2.1:1.

Paragraph 63. The pharmaceutical combination for use according to any one of Paragraphs 51 to 59, wherein the molar ratio of the taxane anticancer drug or a pharmaceutically acceptable salt thereof (e.g. paclitaxel) to the compound of formula (I) or a pharmaceutically acceptable salt thereof per weekly dose is e.g. about 0.6:1 to about 0.9:1.

Paragraph 64. The pharmaceutical combination for use according to any one of Paragraphs 51 to 59, wherein the compound of formula (I) or a pharmaceutically acceptable salt thereof is administered to a patient at a dose corresponding to the mole equivalent of about 5 mg to about 480 mg of the free base of the compound of formula (I) per week in weeks when administered.

Paragraph 65. The pharmaceutical combination for use according to any one of Paragraphs 51 to 59, wherein the compound of formula (I) or a pharmaceutically acceptable salt thereof is administered to a patient at a dose corresponding to the mole equivalent of about 40 mg to about 200 mg of the free base of the compound of formula (I) per week in weeks when administered.

Paragraph 66. The pharmaceutical combination for use according to any one of Paragraphs 51 to 59, wherein the compound of formula (I) or a pharmaceutically acceptable salt thereof is administered to a patient at a dose corresponding to the mole equivalent of about 80 mg to about 160 mg of the free base of the compound of formula (I) per week in weeks when administered.

Paragraph 67. The pharmaceutical combination for use according to any one of Paragraphs 51 to 59, wherein the compound of formula (I) or a pharmaceutically acceptable salt thereof is administered to a patient at a dose corresponding to the mole equivalent of about 90 mg to about 130 mg of the free base of the compound of formula (I) per week in weeks when administered.

Paragraph 68. The pharmaceutical combination for use according to any one of Paragraphs 51 to 59, wherein the compound of formula (I) or a pharmaceutically acceptable salt thereof is administered to a patient at a dose corresponding to the mole equivalent of about 140 mg to about 240 mg of the free base of the compound of formula (I) per week in weeks when administered.

Paragraph 69. The pharmaceutical combination for use according to any one of Paragraphs 51 to 59, wherein the compound of formula (I) or a pharmaceutically acceptable salt thereof is administered to a patient at a dose corresponding to the mole equivalent of about 160 mg to about 220 mg of the free base of the compound of formula (I) per week in weeks when administered.

Paragraph 70. The pharmaceutical combination for use according to any one of Paragraphs 51 to 59, wherein the compound of formula (I) or a pharmaceutically acceptable salt thereof is administered to a patient at a dose corresponding to the mole equivalent of about 180 mg to about 200 mg of the free base of the compound of formula (I) per week in weeks when administered.

Paragraph 71. The pharmaceutical combination for use according to any one of Paragraphs 64 to 70, wherein the taxane anticancer drug or a pharmaceutically acceptable salt thereof is paclitaxel and paclitaxel is administered to a patient at a dose of about 60 mg/m² to about 90 mg/m² per week in weeks when administered.

Paragraph 72. The pharmaceutical combination for use according to any one of Paragraphs 64 to 70, wherein the taxane anticancer drug or a pharmaceutically acceptable salt thereof is paclitaxel and paclitaxel is administered to a patient at a dose of about 60 mg/m² to about 80 mg/m² per week in weeks when administered.

Paragraph 73. The pharmaceutical combination for use according to any one of Paragraphs 64 to 70, wherein the taxane anticancer drug or a pharmaceutically acceptable salt thereof is paclitaxel and paclitaxel is administered to a patient at a dose of about 70 mg/m² to about 80 mg/m² per week in weeks when administered.

Paragraph 74. The pharmaceutical combination for use according to any one of Paragraphs 51 to 73, wherein the compound of formula (I) or a pharmaceutically acceptable salt thereof and the taxane anticancer drug or a pharmaceutically acceptable salt thereof (e.g. paclitaxel) are administered on the same day and wherein the compound of formula (I) or a pharmaceutically acceptable salt thereof is administered to the subject before the taxane anticancer drug or a pharmaceutically acceptable salt thereof (e.g. paclitaxel).

Paragraph 75. The pharmaceutical combination for use according to any one of Paragraphs 51 to 73, wherein the compound of formula (I) or a pharmaceutically acceptable salt thereof and the taxane anticancer drug or a pharmaceutically acceptable salt thereof (e.g. paclitaxel) are administered on the same day and wherein the compound of formula (I) or a pharmaceutically acceptable salt thereof is administered to the subject after the taxane anticancer drug or a pharmaceutically acceptable salt thereof (e.g. paclitaxel).

Paragraph 76. The pharmaceutical combination for use according to Paragraph 74 or Paragraph 75, wherein the time interval between the administrations is at least about 4 hours.

Paragraph 77. The pharmaceutical combination for use according to Paragraph 74 or Paragraph 75, wherein the time interval between the administrations is at least about 2 hours.

Paragraph 78. The pharmaceutical combination for use according to any one of Paragraphs 51 to 77, wherein the neoplastic disease is a solid tumor.

Paragraph 79. The pharmaceutical combination for use according to any one of Paragraphs 51 to 78, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, miscellaneous myeloproliferative disorders, lymphoproliferative disorders and myelodysplastic syndromes.

Paragraph 80. The pharmaceutical combination for use according to any one of Paragraphs 51 to 79 wherein the neoplastic disease one which is treatable by inhibition of PLK1 in addition to a treatment with a taxane anticancer drug (e.g. paclitaxel) and a TTK inhibitor (e.g. the compound of formula (I)).

Paragraph 81. The pharmaceutical combination for use according to any one of Paragraphs 51 to 80, wherein the neoplastic disease is cancer.

Paragraph 82. The pharmaceutical combination for use according to Paragraph 81, wherein the cancer in terms of the organs and parts of the body affected is selected from the brain, breast (including triple negative breast cancer and luminal B breast cancer), cervix, ovaries, colon, rectum (including colon and rectum i.e. colorectal cancer), lung (including small cell lung cancer, non-small cell lung cancer, large cell lung cancer and mesothelioma), endocrine system, bone, adrenal gland, thymus, liver, stomach, intestine (including gastric cancer), pancreas, bone marrow, hematological malignancies (such as lymphoma, leukemia, myeloma or lymphoid malignancies), bile duct, bladder, urinary tract, kidneys, skin, thyroid, head, neck, prostate and testis.

Paragraph 83. The pharmaceutical combination for use according to Paragraph 81, wherein the cancer is selected from breast cancer (including triple negative breast cancer and luminal B cancer), gastric cancer, colorectal cancer, liver cancer (including hepatocellular cancer), endometrial cancer, ovarian cancer, esophageal cancer, lung cancer (including non-small cell lung cancer), Kaposi's sarcoma, cervical cancer, pancreatic cancer, melanoma, prostate cancer and bladder cancer and leukemia, e.g. acute myeloid leukemia (AML) (including Complex Karyotype AML).

Paragraph 84. The pharmaceutical combination for use according to Paragraph 81, wherein the cancer is breast cancer.

Paragraph 85. The pharmaceutical combination for use according to Paragraph 81, wherein the cancer is triple negative breast cancer.

Paragraph 86. The pharmaceutical combination for use according to Paragraph 81, wherein the cancer is luminal B breast cancer.

Paragraph 87. A compound of formula (I) or a pharmaceutically acceptable salt thereof as defined in Paragraph 1 for use in combination with a taxane anticancer drug or a pharmaceutically acceptable salt thereof as defined in any one of Paragraphs 1 to 3 (e.g. paclitaxel) for the treatment of a neoplastic disease in a subject, in particular a human.

Paragraph 88. The compound of formula (I) or a pharmaceutically acceptable salt thereof for use according to Paragraph 87, wherein the compound of formula (I) or a pharmaceutically acceptable salt thereof is administered to the subject as defined in any one of Paragraphs 53 to 77.

Paragraph 89. The compound of formula (I) or a pharmaceutically acceptable salt thereof for use according to Paragraph 78 or Paragraph 79, wherein the neoplastic disease is as defined in any one of Paragraphs 78 to 86.

Paragraph 90. A taxane anticancer drug or a pharmaceutically acceptable salt thereof as defined in any one of Paragraphs 1 to 3 (e.g. paclitaxel) for use in combination with a compound of formula (I) or a pharmaceutically acceptable salt thereof as defined in Paragraph 1 for the treatment of a neoplastic disease in a subject, in particular a human.

Paragraph 91. The taxane anticancer drug or a pharmaceutically acceptable salt thereof for use according to Paragraph 90, wherein the taxane anticancer drug or a pharmaceutically acceptable salt thereof (e.g. paclitaxel) is administered to the subject as defined in any one of Paragraphs 53 to 77.

Paragraph 92. The taxane anticancer drug or a pharmaceutically acceptable salt thereof for use according to Paragraph 90 or Paragraph 91, wherein the neoplastic disease is as defined in any one of Paragraphs 78 to 86.

Paragraph 93. Use of a pharmaceutical combination as defined in any one of Paragraphs 1 to 8 in the preparation of single-agent medicaments or as a combined medicament for the treatment of a neoplastic disease in a subject, in particular a human.

Paragraph 94. The use according to Paragraph 93, wherein the pharmaceutical composition is administered to the subject as defined in any one of Paragraphs 53 to 77.

Paragraph 95. The use according to Paragraph 93 or Paragraph 94, wherein the neoplastic disease is as defined in any one of Paragraphs 78 to 86.

Paragraph 96. Use of a compound of formula (I) or a pharmaceutically acceptable salt thereof as defined in Paragraph 1 in the preparation of a single-agent medicament for use in combination with a taxane anticancer drug or a pharmaceutically acceptable salt thereof as defined in any one of Paragraphs 1 to 3 (e.g. paclitaxel), or in the preparation of a combined medicament with a taxane anticancer drug or a pharmaceutically acceptable salt thereof in any one of Paragraphs 1 to 3 (e.g. paclitaxel), for the treatment of a neoplastic disease in a subject, in particular a human.

Paragraph 97. The use according to Paragraph 96, wherein the compound of formula (I) or a pharmaceutically acceptable salt thereof is administered to the subject as defined in any one of Paragraphs 53 to 77.

Paragraph 98. The use according to Paragraph 96 or Paragraph 94, wherein the neoplastic disease is as defined in any one of Paragraphs 78 to 86.

Paragraph 99. Use of a taxane anticancer drug or a pharmaceutically acceptable salt thereof as defined in any one of Paragraphs 1 to 3 (e.g. paclitaxel) in the preparation of a single-agent medicament for use in combination with a compound of formula (I) or a pharmaceutically acceptable salt thereof as defined in Paragraph 1 or in the preparation of a combined medicament with the compound of formula (I) or a pharmaceutically acceptable salt thereof as defined in Paragraph 1, for the treatment of a neoplastic disease in a subject, in particular a human.

Paragraph 100. The use according to Paragraph 99, wherein the taxane anticancer drug or a pharmaceutically acceptable salt thereof (e.g. paclitaxel) is administered to the subject as defined in any one of Paragraphs 53 to 77.

Paragraph 101. The use according to Paragraph 99 or Paragraph 100, wherein the neoplastic disease is as defined in any one of Paragraphs 78 to 86.

Paragraph 102. A kit comprising a pharmaceutical combination as defined in any one of Paragraphs 1 to 8, wherein component (a) and component (b) are provided as single agent medicaments.

Paragraph 103. The kit according to Paragraph 102, wherein the kit is for use in treating a neoplastic disease, in particular as defined in any one of Paragraphs 78 to 86.

Paragraph 104. The kit according to Paragraph 102 or Paragraph 103, further comprising instructions for simultaneous, separate or sequential administration thereof for use in the treatment of a neoplastic disease, in particular a cancer, in a subject, in particular a human.

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Particular embodiments of the invention are described in the following Examples, which serve to illustrate the invention in more detail and should not be construed as limiting the invention in any way.

### EXAMPLES

### Methodology

### Kinase Assay

A radiometric protein kinase assay (33PanQinase® Activity Assay) was used for measuring the kinase activity of TTK and PLK1. TTK and PLK1 protein kinases were expressed as recombinant full-length GST-fusion proteins. The reaction cocktail contained 25 mL of assay buffer (standard buffer/[γ-33P]-ATP) and 10 mL of ATP solution (in water), 5 mL of test compound and 10 mL of enzyme/substrate mixture. The assay for the protein kinases contained 70 mM HEPES-NaOH pH 7.5, 3 mM MgCl2, 3 mM MnCl2, 3 mM Na-orthovanadate, 1.2 mM DTT, 50 mg/ml PEG20000, ATP (0.3 mM for TTK and 1 mM for PLK1), [γ-33P]-ATP (approx. 8×105 cpm per well), protein kinase (15.8 nM for TTK and 5 nM for PLK1), and substrate (1 mg/50 mL for TTK and 2 mg/50 mL for PLK1). All assays were performed with a Beckman-Coulter/SAGIAN™ Core System. The fitting model for the IC50 determinations was “Sigmoidal response (variable slope)” with parameters “top” fixed at 100% and “bottom” at 0%. The fitting method used was a least squares fit.

### Target Residence Time Assays

The affinity, i.e. the equilibrium dissociation constant (KD) (referred to as residence times), of the compound of formula (I) against TTK and PLK1 were determined using a Biacore T200™ surface plasmon resonance instrument using recombinant expressed TTK kinase domain (amino acids 519-808) or biotinylated PLK1. The immobilization of TTK was performed as described in Maia et al. Annals of Oncology, 2015; 26:2180-2192. The immobilization of biotinylated PLK1 was performed as described in Willemsen-Seegers et al. Journal of Molecular Biology, 2017; 429:574-586. The subsequent single cycle kinetic assays were performed at 22° C. using a compound concentration gradient of 1, 3.6, 10, 31.6 and 100 nM for TTK and 10, 31.6, 100, 316 and 1000 nM for PLK1, a contact time of 100 s and a flow rate of 30 mL/min. The dissociation period was 1200-1800 s and a correction for an unstable surface using a blank run with buffer was performed. Binding kinetics were calculated based on the binding curves, demonstrating good signal-to-noise ratios for all compounds tested (data not shown).

### PDX Studies

In a first PDX study female BALB/c nude mice (CrownBio) of at least 20 g body weight were subcutaneously (s.c.) inoculated in the right flank with tumor fragments (3*3*3 mm) from stock mice bearing the patient-derived xenograft (PDX) triple negative breast cancer (TNBC) tumor, BR1282. Randomization into 5 different treatment groups (8 mice per group) was made when the mean tumor size was 150-200 mm3. Mice were dosed intravenously (i.v.) twice weekly (2qw) with the compound of formula (I) (8 mg/kg, i.v., 5 mL/kg) or once weekly (qw) paclitaxel (15 mg/kg, i.v., 10 mL/kg) or the combination of these doses, in which the compound of formula (I) was administered after paclitaxel using either a 4 h or a 24 h gap, when both compounds were administered on the same day/consecutive days. The fifth group received both vehicles using the time interval of 4 h and all groups were dosed twice via the tail vein. Dosing was delayed by 1 day in any mice showing transient body weight loss of ≥ 10% at any time point. Treatments continued until day 35/36, when it was necessary to cease administration because of toxicity (necrosis) at the site of injection in the tail vein. At that point an extended observation period followed for the remaining two combination groups (culling due to large tumor volumes unnecessary).

In a second PDX study, the same approach and model was used as for the first study (TNBC BR1282 PDX model), but this time additional combinations were tested, including the opposite order of administration (i.e. treating with the compound of formula (I) followed by paclitaxel) as well as use of a qw schedule regimen for compound of formula (I) as well as a 2qw regimen as used before. Mice (8 mice per group) were dosed with either the compound of formula (I) (8 mg/kg, i.v., 5 mL/kg), paclitaxel (15 mg/kg, i.v., 10 mL/kg, qw), a combination of both compounds or both vehicles as control group. The single agent group of compound of formula (I) was treated first with the vehicle of paclitaxel (qw, 10 mL/kg) and 4 h later, when treated on the same day, with the compound of formula (I), which was administered 2qw. The paclitaxel single agent group received paclitaxel (15 mg/kg, i.v., qw, 10 mL/kg) followed by the vehicle of compound of formula (I) with a 2 h gap between the administrations. The five different combination groups received the following dosing: paclitaxel (15 mg/kg, i.v., qw, 10 mL/kg) administered first and compound of formula (I) (8 mg/kg, i.v. 2qw, 5 mL/kg) second using a 2 h, 4 h or 24 h gap between both administrations when administered on the same or on consecutive days; paclitaxel (15 mg/kg, i.v., qw, 10 mL/kg) administered first and compound of formula (I) (8 mg/kg, i.v. qw, 5 mL/kg) second using a 4 h gap and always treated on the same day; and finally compound of formula (I) (8 mg/kg, i.v., 2qw, 5 mL/kg) administered first and paclitaxel (15 mg/kg, i.v., qw, 5 mL/kg) second using a 4 h gap. The eighth group received both vehicles using a 4 h gap with the vehicle of paclitaxel administered first. Dosing was delayed by 1 day in any mice showing transient body weight loss of ≥ 10% at any time point. Treatments continued until day 42/43, when it was necessary to cease administration because of toxicity (necrosis) at the site of injection in the tail vein. At that point, the four combination groups and the 4 h gap single agent group of compound of formula (I) (culling due to large tumor volumes unnecessary) were observed for potential cures and tumor regrowth (study not complete).

Paclitaxel was purchased from Beijing Union Pharmaceutical Factory as a solution (6 mg/mL in anhydrous citrate, polyoxyethylated (35) castor oil and absolute ethyl alcohol). The compound of formula (I) was supplied as a powder by Basilea Pharmaceutica International Ltd. and was prepared freshly as a solution in Ethanol: PEG400:Citric acid 20 mM, at a ratio of 1:1:8, for immediate administration.

Body weights and tumor volumes were determined at least twice per week, the latter by measuring two dimensions with calipers and applying the formula “V=(L×W2)/2”, where V is the tumor volume, and L and W are the tumor length and width respectively. Individual mice were culled when tumors reached 1500 mm3 or more, or when the body weight loss (BWL) was found to have exceeded 20%. Mice were also culled if the BWL was determined to be >15% for 3 consecutive days. Any mice with >10% BWL, automatically were dosed on one of the following days, as soon as the BWL returned to <10%. All animal protocols were reviewed and approved by the relevant local committees in China, where the studies were performed.

The statistical significance of efficacy were determined in two different ways using a) the endpoint on day 16 or day 13 depending on the study when it was necessary to cull the vehicle group because of tumor volume exceeding 1500 mm3, and b) at the endpoint of day 42 or day 51 depending on the study (one week after the final treatment) when exponential curve fits were applied to estimate the rates of tumor growth in the different groups. Finally, the number of complete cures was compared across the groups following confirmation by histopathology within the first study. The degree of an effect was quantified by the delta (Δ) treated/control ratio (ΔT/C) and the statistical significance of the effect was determined by a one-way analysis of variance (1W-ANOVA) with Holms-Sidak applied post-hoc for multiple determinations, where a p-value of <0.05 was considered statistically significant. In addition, an assessment of synergy was made based upon the Clarkes Combination Index (CCI), where the CCI=ΔT/CAB−(ΔT/CA*ΔT/CB) in which A and B are two different compounds, where CCI<−0.1 indicates synergy (or a positive interaction), +0.1 (antagonism, or a negative interaction) and between −0.1 and +0.1 indicates additivity (no interaction); see O'Reilly et al. Anti Cancer Drugs 2011; 22:58-78.

### Results

TTK and PLK1 are kinases with an essential role in control of the spindle assembly checkpoint (SAC), which is a cell cycle surveillance mechanism ensuring optimal cell division via proper chromosome alignment. TTK and PLK1 co-operate to recruit SAC components to the SAC protein complex at the kinetochore of the chromosome, thus inhibition of both enzymes should maximize progression of mitosis via more rapid breakage of the SAC (Von Schubert et al., Cell Reports 2015, 12; 66-78). This has proven to be the case in tumor cell systems when comparing the compound of formula (I) with TKK inhibitors not having any meaningful PLK1 inhibitory activity (data not shown).

In general, the compound of formula (I) shows strong specificity for TTK, with other kinase IC50s being at least 10-fold higher than those for TTK. The TTK kinase assay described above confirmed that the compound of formula (I) is highly potent against TTK, giving an IC50 of 7 nM (0.4 nM when measured as described in WO 2015/155042).

The PLK1 kinase assay described above also showed that the compound of formula (I) targets PLK1. The compound of formula (I) was found to inhibit PLK1 with an IC50 of 72 nM. When measured as described in WO 2015/155042 the compound of formula (I) inhibited PLK1 with an IC50 of 46 nM. Other TTK inhibitors reported in the literature have similar or slightly better overall specificity for TTK but conversely have little or no activity against PLK1 relative to their activity against TTK (data not shown). Importantly, the compound of formula (I) has a very long target-residency of >12 h on TTK, while that for PLK1 is just a few minutes. This prolonged inhibition of TTK combined with a transient effect on PLK1 leads to a rapid disruption of the SAC leaving the cells without adequate time for correct chromosome segregation.

Prolonged TTK target occupancy was also measured in tumors derived from MDAMB-231 xenograft-bearing mice treated with intermittent i.v. dosing regimen of compound of formula (I). To determine the tumor TTK target occupancy time of the compound of formula (I), mice harboring the TNBC xenograft model MDA-MB-231 were treated i.v. twice-weekly with MTD and sub-MTD doses of the compound of formula (I). Analysis of vehicle- and compound of formula (I)-treated tumors for TTK target occupancy indicated that the compound of formula (I) occupied tumor-derived TTK in a concentration-dependent manner; TTK was completely occupied by the compound of formula (I) for at least 72 h after administration of the last MTD dose. A repeat experiment using i.v., weekly MTD dosing indicated that tumor-derived TTK was fully drug-occupied for up to 6 days after the last administration.

In cellular systems, the compound of formula (I) has high potency against sensitive cells. In a 5-day anti-proliferative screen of 18 different triple negative breast cancer cell lines, the compound of formula (I) had a median GI50 of 35 nM. In mice, the compound of formula (I) has shown significant activity against patient-derived tumor xenografts, including TNBC and hepatocellular cancer (HCC) models, with effects ranging from minimal to very strong including substantial regression (data not shown).

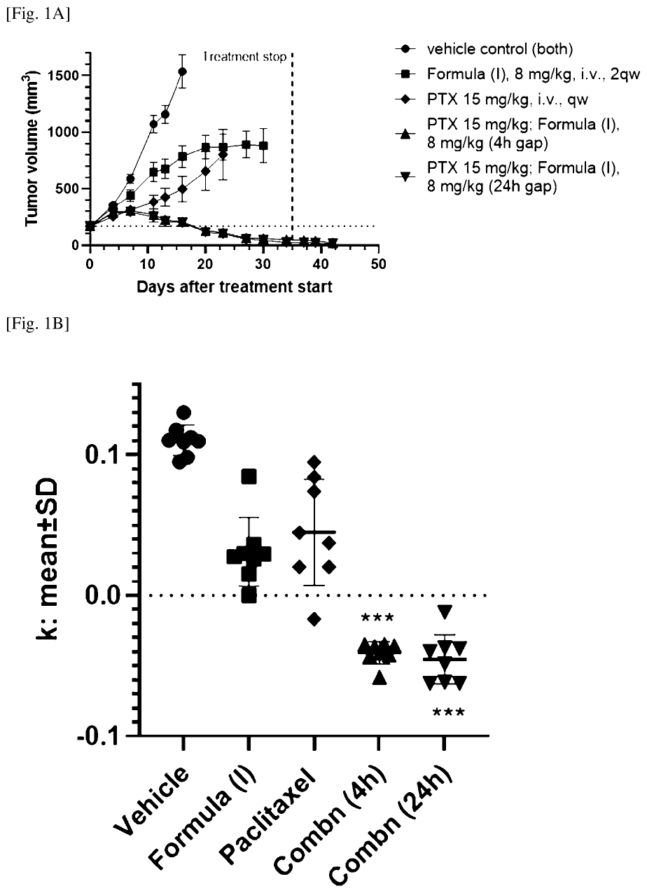

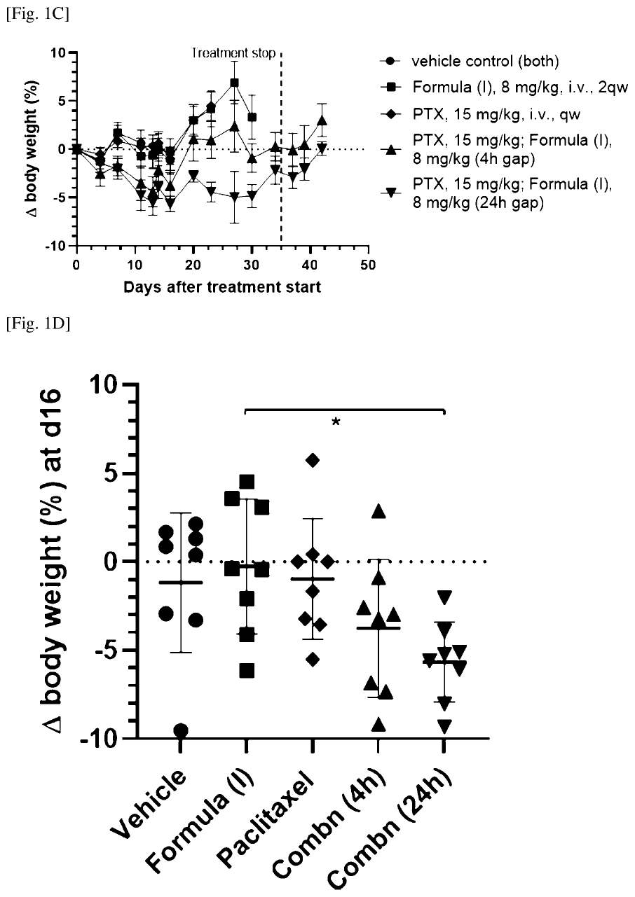

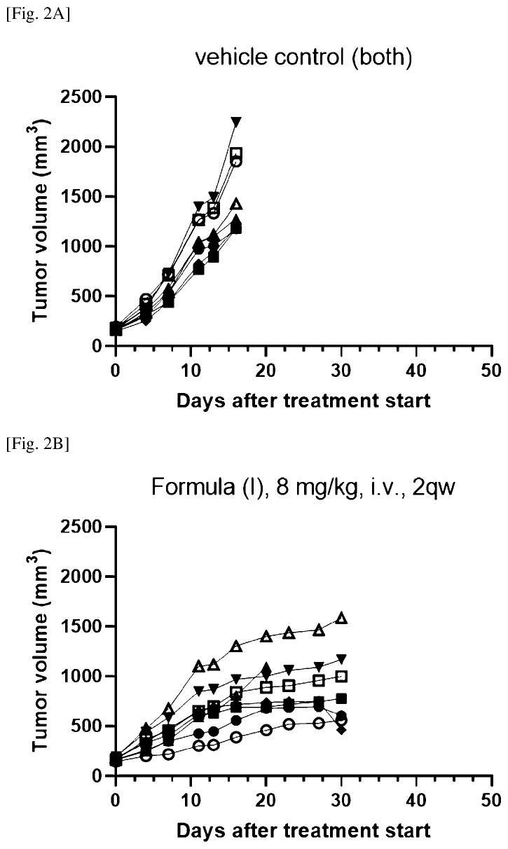

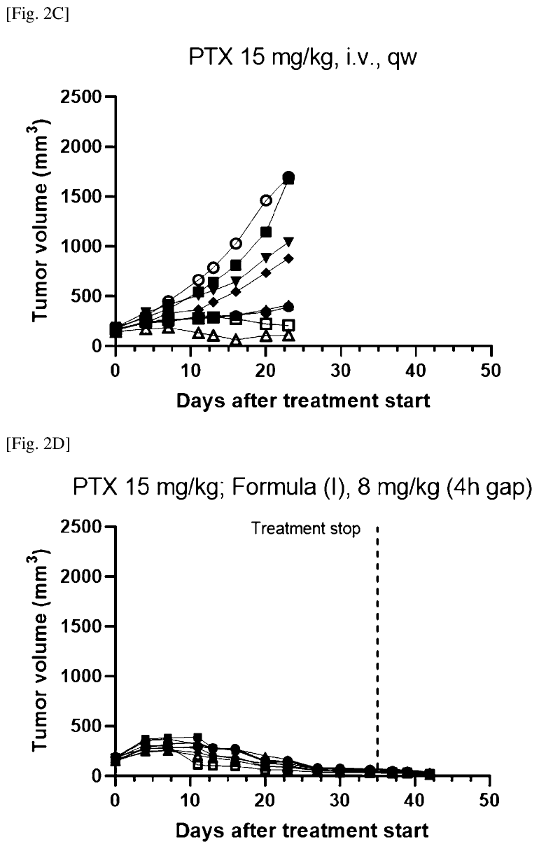

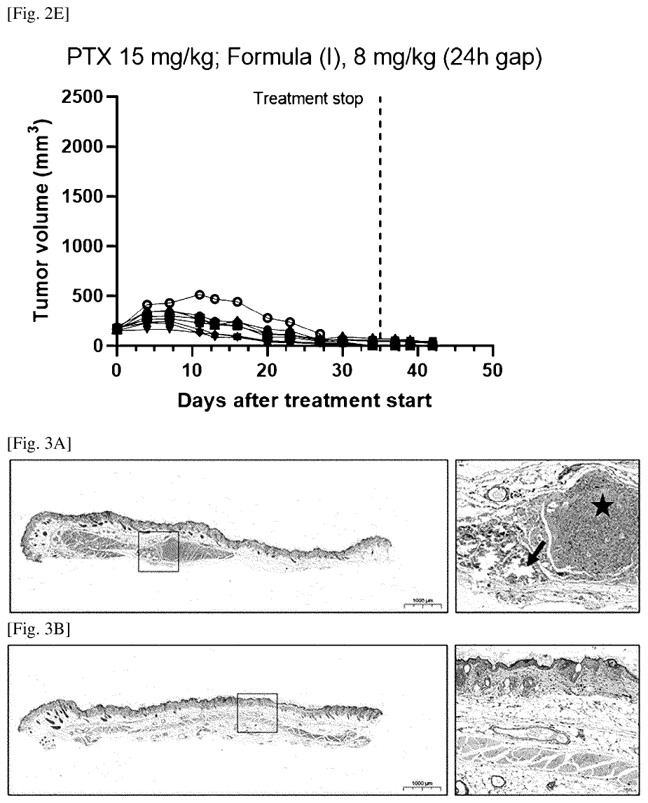

In the first PDX study the TNBC BR1282 model grew rapidly and thus the vehicle group required culling after 16 days (FIG. 1). At that time point, it was apparent that all treatments had some level of efficacy (Table 1) and indeed, both monotherapies and both combination treatments were highly significantly different to the vehicle (all p<0.001). The combinations were also significantly different to the compound of formula (I) alone (p<0.001), but not paclitaxel alone (p=0.12). The CCIs indicated a positive interaction with values of −0.08 and −0.09 for the 4 h and 24 h combination groups respectively, although not quite formal synergy at that time point (defined as <−0.1). The monotherapy groups continued to grow and also had to be culled by day 30, while the combination groups continued to regress and by day 42 (one week after treatment ceased, i.e. one treatment cycle), all animals had regressed with means±SD of −87±6% and −92±11% for the combination 4 h and combination 24 h groups respectively (see also FIG. 2).

Exponential curve fits from day 0 until culling day or day 42, provided more statistical power and showed a much stronger effect, so that both combination groups were now highly significantly different to either monotherapy (FIG. 1B), and the CCI indicated strong synergies of −0.49 and −0.53 for the 4 h and 24 h combination groups respectively. Following an extended observation period without treatment pathological evaluation by H&E staining of the skin around the site of tumor cell injection of tumor-free animals indicated cures in 3 out of 8 treated animals in both combination groups. Examples of the H&E staining results are shown in FIG. 3.

Overall, the treatments were well tolerated with no significant differences in body weight change for the treatment groups compared to the vehicle group at day 16. Thereafter, the monotherapy groups had body weight gain, as did the combination 4 h group, but the combination 24 h group showed transient body weight loss with recovery by day 35. Once treatment ceased, there was strong body weight recovery by both combination groups. The combination 24 h group also had 1/8 mice that had to be culled (day 28) due to a body weight loss of 20%. Thus, the longer gap between the two treatments in the combination therapy did seem to be slightly less tolerated in this experiment.

In conclusion, in the first PDX study the compound of formula (I) combined with paclitaxel produced a very strong positive interaction inducing 100% tumor regression, which included 3/8 complete cures in each of the two combination groups which differed only in the time interval (4 h or 24 h gap) between dosing paclitaxel (first) and the compound of formula (I) (second). The combination 24 h group appeared to be slightly less tolerated shown by slightly higher, albeit transient, body weight loss.

TABLE 1
Summary of efficacy and tolerability in the first combination

| | Efficacy: ΔT/C | | | | |
|---|---|---|---|---|---|
| | | Day −0 | Cures | | |
| | Day | up to | Day | Tolerability (Day 16) | |
| Treatment | 16 | Day 42 | 97 | ΔBW (%) | ΔT/C |
| Vehicles | 1.0 | 1.0 | 0/8 | −1.2 ± 1.4 | 1.0 |
| Compound of | 0.45 | 0.28*** | 0/8 | −0.3 ± 1.4 | 1.01 |
| formula (I) | | | | | |
| Paclitaxel | 0.24 | 0.41*** | 0/8 | −1.0 ± 1.2 | 1.00 |
| Combn-4 h | 0.03 | −0.37*** | 3/8 | −3.8 ± 1.4 | 0.97 |
| Combn-24 h | 0.02 | −0.41*** | 3/8 | −5.7 ± 0.8 | 0.95 |

Results for efficacy show the calculated ΔT/Cs based on the mean change in tumor volume for each group on day 16 and the mean rate of growth (k) measured from day 0 to day 42 (one week after all treatment ceased).
Growth rate (k) is calculated based on the time period for each treatment, i.e. D 0-D 16 (Vehicle), D 0-D 37 for the monotherapies and D 0-D 42 for the two combination groups.
All treatments were highly significantly different to the vehicle group, where
***p < 0.001 (one way ANOVA with Holm-Sidak post-hoc).
The number of cures (no tumors detectable by H&E staining) for each treatment group is shown based upon H&E staining of skin on day 97.
Results for tolerability show the mean ± SEM for the percentage change in body weight (BW) on day 16 and the ΔT/C based on the mean fractional change compared to the vehicle (BW_{day 13}/BW_{day 0}).

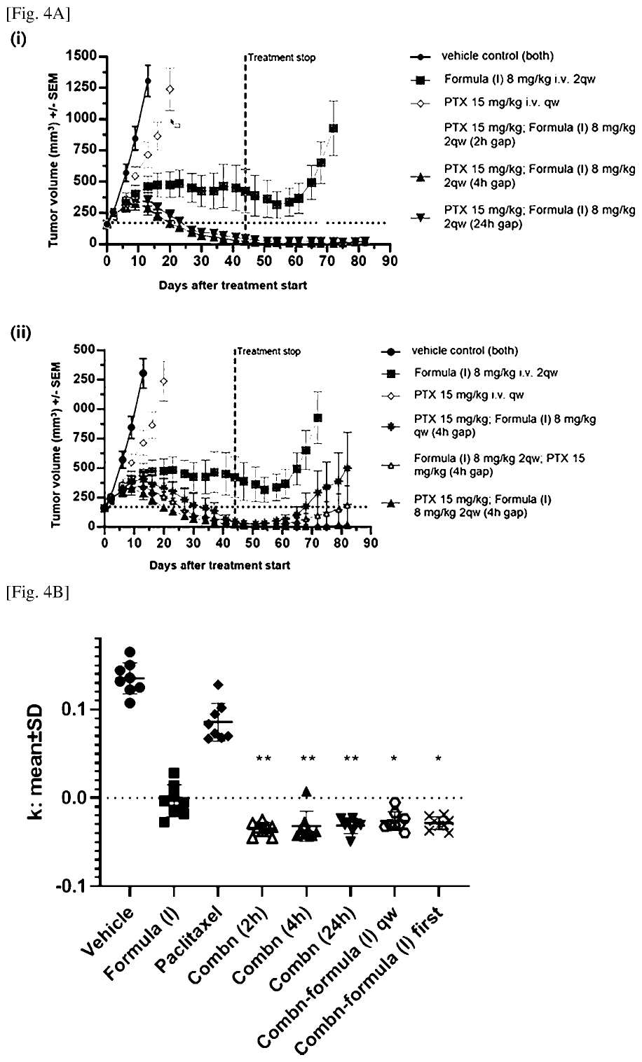

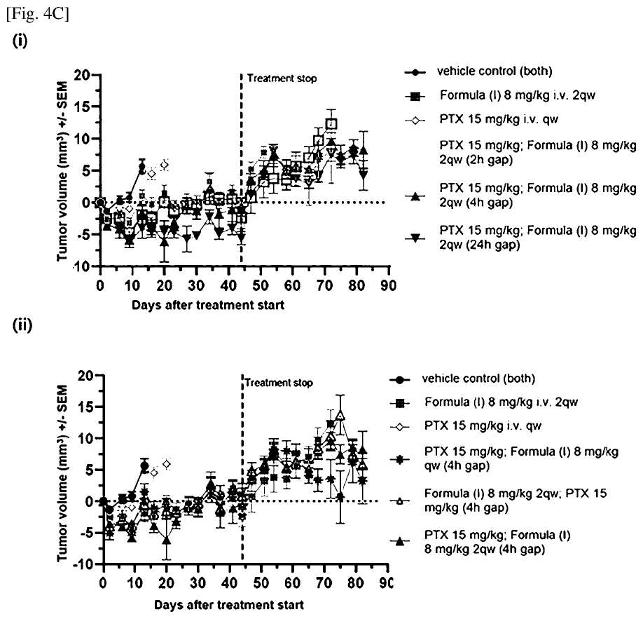

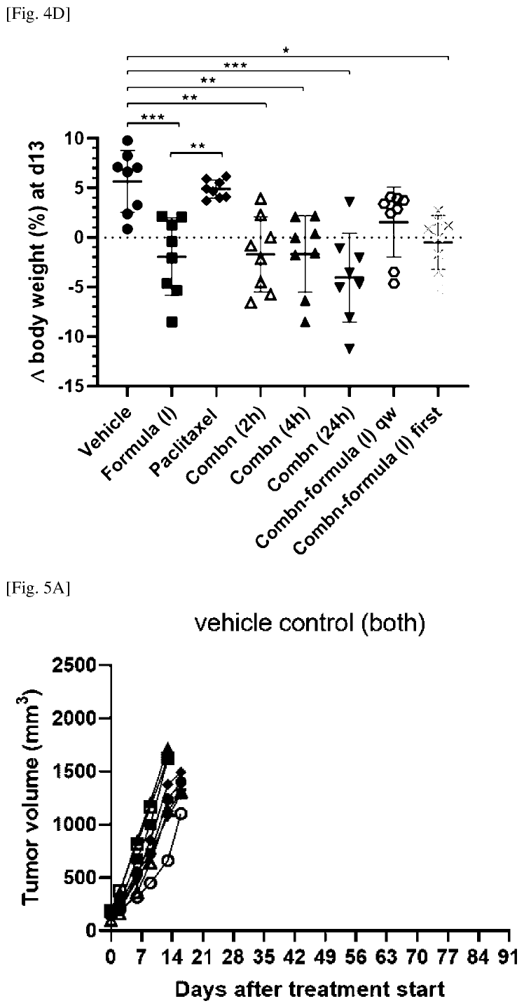

In the second study, the vehicle control group reached the ethical endpoint on day 13. At that time point it was apparent that all treatments had some level of efficacy (Table 2, FIG. 4A) and indeed, both monotherapies and all combination treatments were highly significantly different to the vehicle (all p<0.001). The combinations were also significantly different to paclitaxel alone (p<0.01), but not to the compound of formula (I) alone (p≥0.78). The CCIs indicated a positive interaction with values between −0.02 and −0.09 (Table 3). The monotherapy groups continued to grow and the group treated with paclitaxel alone had to be culled by day 20, while the combination groups continued to regress and by day 51 (roughly one week after treatment ceased, i.e. one treatment cycle) all animals had regressed with the following means±SD: −86±15% for the combination 2 h group, −94±8% for the combination 4 h group, −86±8% for the combination 24 h group, −84±26% for the combination group using compound of formula (I) qw, and −86±10% for the combination group administering compound of formula (I) before paclitaxel. Although 3/8 animals after monotherapy of compound of formula (I) indicated regression on that day as well, the overall group mean (or median) did not show regression, but tumor growth (FIG. 4A).

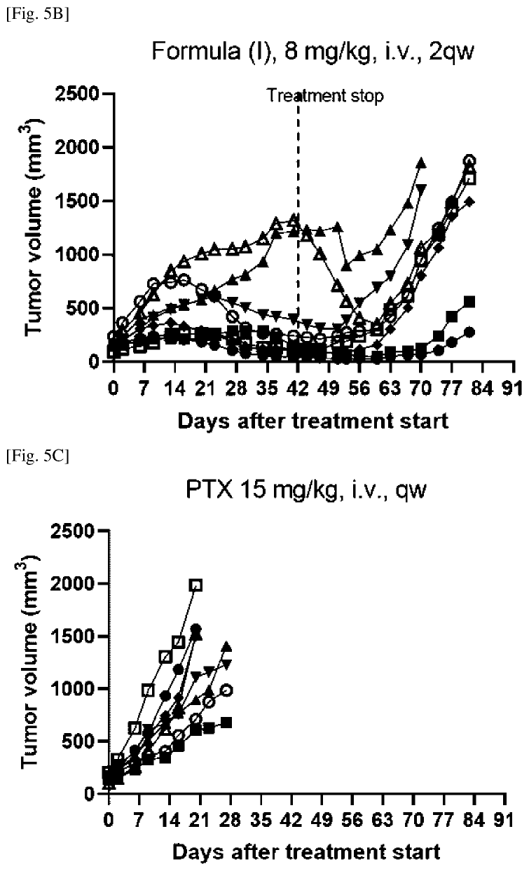

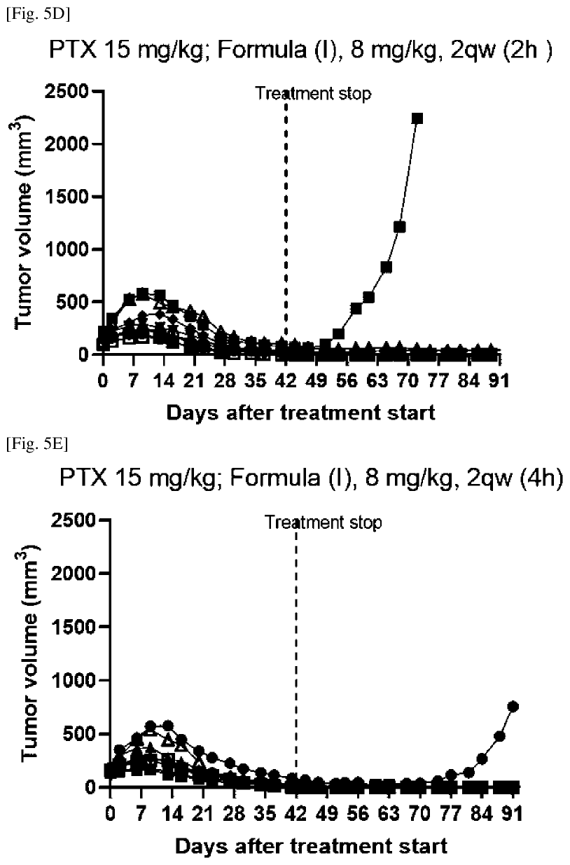

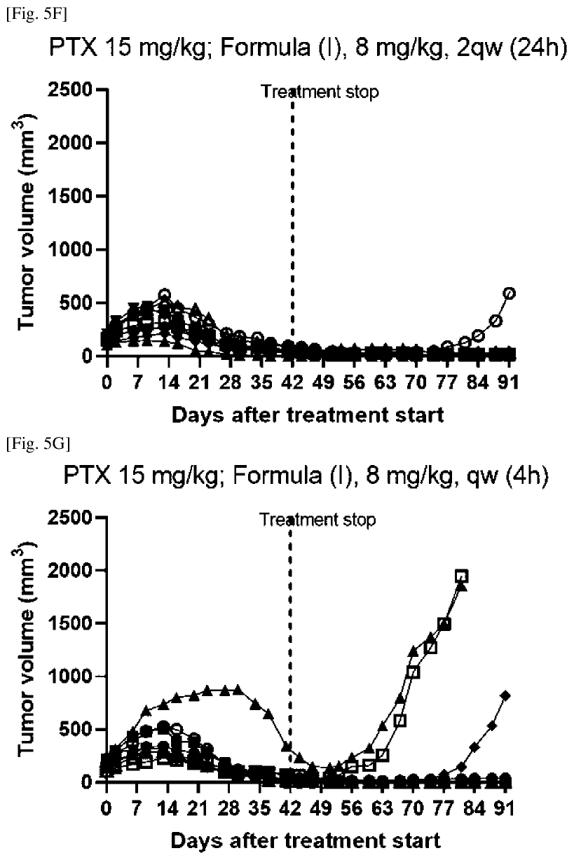

The individual tumor growth curves also demonstrated that the tumors of the compound of formula (I) monotherapy group started to regrow earlier and faster than any of the combination groups (FIG. 5). The highly synergistic effect of all the combination groups confirmed and extended previous results from the first study. Exponential curve fits from day 0 until culling day or day 51 (roughly one week after treatment stopped), provided more statistical power and showed a much stronger effect so that all five combination groups were now significantly different to either monotherapy (FIG. 4B) and the CCI indicated synergies between −0.18 and −0.29 (Table 3). FIG. 5 shows no tumor regrowth in any of the combination groups before day 51. After day 51, 1 out of 8 tumors in each combination group, apart from the combination group using compound of formula (I) qw which showed 3 out of 8, did show some regrowth.

Overall, the treatments were well tolerated with no significant differences in body weight change for the treatment groups compared to the vehicle group at day 13 (FIGS. 4C and D, Table 2). Thereafter, the monotherapy groups had body weight gain, as did all combination groups, but the combination 24 h group showed transient body weight loss with recovery by day 42. Once treatment ceased, there was strong body weight recovery by all combination groups. Thus, the longer gap between the two treatments in the combination therapy did seem to be slightly less tolerated in this experiment as well as in the first experiment.

Interestingly, at the end of the observation period (at the latest day 148) 29 mice overall from the combination groups had either no palpable tumor mass detected or a tumor volume of <25 mm³, indicating potential cures. Hence, skin samples from these 29 mice, including the tumor cell injection site and surrounding tissue, were taken and processed to FFPE blocks. Serial sections covering the entire tumor growth area were produced, stained by H&E and subsequently pathologically evaluated for residual tumor cells. Results are shown in Table 4 and indicate 50-88% cures (4-7 mice out of 8) in the combination groups. Interestingly, qw administration of the compound of formula (I), as well as treating with the compound of formula (I) first and paclitaxel second, resulted in the lowest number of cures (4 and 5 out of 8 mice, respectively). Administering paclitaxel first followed by the compound of formula (I) 2, 4 or 24 h later on a 2qw schedule led to cures in 6-7 mice out of 8. Consequently, the latter treatment regimens seemed to be the most efficacious with regard to number of cures.

In conclusion, in the second PDX study the compound of formula (I) combined with paclitaxel produced a very strong positive interaction inducing 100% tumor regression independent of the dosing regimen used. Treatment was stopped on day 42/43, followed by an extended observation period until at the latest day 148. After this time, 29 animals of the combination groups were pathologically confirmed as cured.

The combination groups differed in the time interval (2 h, 4 h or 24 h gap) between dosing paclitaxel (first) and the compound of formula (I) (second), in the dosing interval of compound of formula (I) (2qw versus qw) or in the order of administration. Although the single agent treatment of compound of formula (I) resulted in this study in tumor regressions, the effect of combining it with paclitaxel resulted in stronger regressions and led to tumor-free animals (including cures) which was not the case for the single agent treatment groups. All five combination regimens were similarly efficacious with regards to tumor growth, although the combination using a 24 h gap seemed to be less well tolerated than the other combination groups. These results demonstrate the potential for flexibility in the timing and order of administration of this combination for future clinical studies, and support the approach of combining compound of formula (I) with paclitaxel for the treatment of TNBC patients.

TABLE 2
Summary of efficacy and tolerability in the second PDX study

| | Efficacy: ΔT/C | | | | |
|---|---|---|---|---|---|
| | | Day 0 | Cures | | |
| | | up to | By day | Tolerability (Day 13) | |
| Treatment | Day 13 | day 51 | 148 | ΔBW (%) | ΔT/C |
| Vehicles | 1.0 | 1.0 | 0/8 | 5.7 ± 1.1 | 1.0 |
| Compound of | 0.32 | −0.02*** | 0/8 | −1.9 ± 1.4 | 0.93 |
| formula (I) | | | | | |
| Paclitaxel | 0.48 | 0.63*** | 0/8 | 4.9 ± 0.3 | 1.02 |
| Combn-2 h | −0.02 | −0.26*** | 7/8 | −1.7 ± 1.3 | 0.98 |
| Combn-4 h | −0.02 | −0.24*** | 6/8 | −1.7 ± 1.4 | 0.93 |
| Combn-24 h | 0.05 | −0.23*** | 7/8 | −4.0 ± 1.6 | 0.96 |
| Combn-formula | 0.09 | −0.19*** | 4/8 | 1.5 ± 1.2 | 1.03 |
| (I) qw | | | | | |
| Combn-formula | 0.04 | −0.21*** | 5/8 | −0.5 ± 1.0 | 0.98 |
| (I) first | | | | | |

Results for efficacy show the calculated ΔT/Cs based on the mean change in tumor volume for each group on day 13 and the mean rate of growth (k) measured from day 0 to day 51 (roughly one week after all treatment ceased).
Growth rate (k) is calculated based on the time period for each treatment, i.e. D 0-D 13 for vehicle, D 0-D 20 for paclitaxel single agent and D 0-D 51 for the five combination groups.
All treatments were highly significantly different to the vehicle group, where
***p < 0.001 (one way ANOVA with Holm-Sidak post-hoc).
The number of cures (no tumors detectable by pathological confirmation) at the end of the observation period for each treatment group is shown.
Results for tolerability show the mean ± SEM for the percentage change in body weight (BW) on day 13 and the ΔT/C based on the mean fractional change compared to the vehicle (BW_{day 13}/BW_{day 0}).

TABLE 3
Summary of the CCI values in the second PDX study

CCI

| Treatment | Day 13 | Day 0 up to day 51 |
|---|---|---|
| Combn-2 h | −0.02 | −0.29 |
| Combn-4 h | −0.02 | −0.22 |
| Combn-24 h | 0.05 | −0.22 |
| Combn-formula (I) qw | 0.09 | −0.18 |
| Combn-formula (I) first | 0.04 | −0.20 |

The CCI was determined as described in Methods

TABLE 4
Summary of pathological evaluation for residual tumor cells

| Dose | Animal no. | Tumor-free | Cures |
|---|---|---|---|
| Combn-2 h | 1 | yes | 87.5% |
| | 3 | yes | (7/8 mice) |
| | 4 | yes | |
| | 5 | yes | |
| | 6 | yes | |
| | 7 | yes | |
| | 8 | yes | |
| Combn-4 h | 2 | yes | 75% |
| | 3 | yes | (6/8 mice) |
| | 4 | yes | |
| | 5 | yes | |
| | 6 | yes | |
| | 7 | yes | |
| Combn-24 h | 1 | yes | 87.5% |
| | 2 | yes | (7/8 mice) |
| | 3 | yes | |
| | 4 | yes | |
| | 5 | yes | |
| | 7 | yes | |
| | 8 | yes | |
| Combn-formula | 2 | yes | 50% |
| (I) qw | 4 | yes | (4/8 mice) |
| | 6 | yes | |
| | 8 | yes | |
| Combn-formula | 1 | yes | 62.5% |
| (I) first | | | (5/8 mice) |

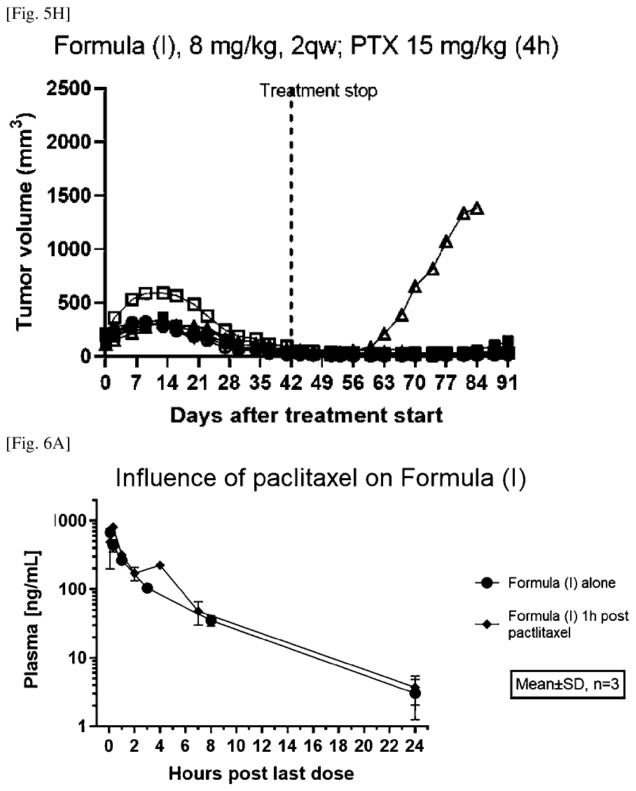

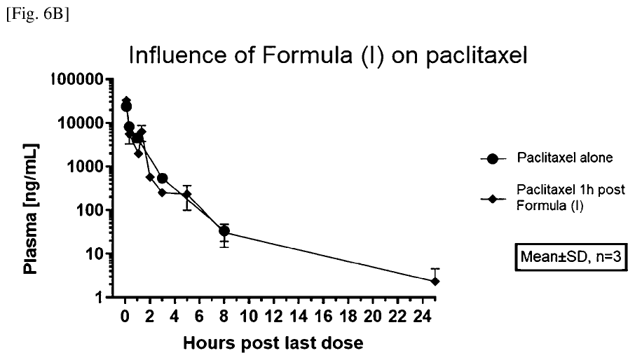

To determine if there was a significant drug-drug interaction (DDI) between the compound of formula (I) and paclitaxel, a single-dose pharmacokinetic (PK) experiment was carried out and the results are shown in FIG. 6. There was a slight nonsignificant increase in the plasma AUC of the compound of formula (I) when it was administered at 8 mg/kg, i.v., 1 h after paclitaxel (15 mg/kg, i.v.). Under the same circumstances, there was also no impact of the compound of formula (I) on the plasma PK of paclitaxel. These results confirm that the synergy observed above is due to the mechanisms-of-action and not due to a change in exposure of one or both agents.

1. A pharmaceutical combination comprising
(a) a compound of formula (I) 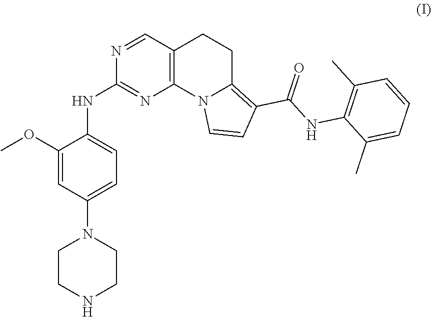 [SMILES: COc1cc(N2CCNCC2)ccc1Nc1ncc2c(n1)-n1ccc(C(=O)Nc3c(C)cccc3C)c1CC2]
or a pharmaceutically acceptable salt thereof; and
(b) a taxane anticancer drug or a pharmaceutically acceptable salt thereof.

2. The pharmaceutical combination of claim 1, wherein the taxane anticancer drug or a pharmaceutically acceptable salt thereof is selected from paclitaxel, docetaxel, cabazitaxel, larotaxel, milataxel, ortataxel and tesetaxel, including pharmaceutically acceptable salts thereof.

3. The pharmaceutical combination of claim 1, wherein the taxane anticancer drug or a pharmaceutically acceptable salt thereof is paclitaxel.

4. The pharmaceutical combination according to claim 1, wherein the molar ratio of the taxane anticancer drug or a pharmaceutically acceptable salt thereof to the compound of formula (I) or a pharmaceutically acceptable salt thereof per weekly dose is about 0.4:1 to about 2.1:1.

5. A method for the treatment of a neoplastic disease in a subject, comprising:
administering to the subject the pharmaceutical combination of claim 1.

6. The method according to claim 5, wherein the compound of formula (I) or a pharmaceutically acceptable salt thereof and the taxane anticancer drug or a pharmaceutically acceptable salt thereof are administered to the subject according to a four week treatment cycle;
wherein the compound of formula (I) or a pharmaceutically acceptable salt thereof is administered in week one of the treatment cycle followed by three rest weeks and the taxane anticancer drug or a pharmaceutically acceptable salt thereof is administered in each week during the first three weeks of the treatment cycle followed by a rest week; or
wherein the compound of formula (I) or a pharmaceutically acceptable salt thereof is administered in week one and in week three of the treatment cycle, with weeks two and four being rest weeks, and the taxane anticancer drug or a pharmaceutically acceptable salt thereof is administered in each week during the first three weeks of the treatment cycle followed by a rest week; or
wherein the compound of formula (I) or a pharmaceutically acceptable salt thereof is administered in each week during the first three weeks of the treatment cycle followed by a rest week, and the taxane anticancer drug or a pharmaceutically acceptable salt thereof is administered in each week during the first three weeks of the treatment cycle followed by a rest week.

7. The method according to claim 5, wherein the compound of formula (I) or a pharmaceutically acceptable salt thereof is administered to a patient at a dose corresponding to the mole equivalent of about 40 mg to about 200 mg of the free base of the compound of formula (I) per week in weeks when administered.

8. The method according to claim 5, wherein the compound of formula (I) or a pharmaceutically acceptable salt thereof is administered to a patient at a dose corresponding to the mole equivalent of about 80 mg to about 160 mg of the free base of the compound of formula (I) per week in weeks when administered.

9. The method according to claim 5, wherein the taxane anticancer drug or a pharmaceutically acceptable salt thereof is paclitaxel and paclitaxel is administered to a patient at a dose of about 70 mg/m 2 to about 80 mg/m 2 per week in weeks when administered.

10. The method according to claim 5, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, myeloproliferative disorders, lymphoproliferative disorders and myelodysplastic syndromes.

11. The method according to claim 5, wherein the neoplastic disease is one which is treatable by inhibition of PLK1 in addition to treatment with a taxane anticancer drug and a TTK inhibitor.

12. The method according to claim 5, wherein the neoplastic disease is a cancer selected from breast cancer, gastric cancer, colorectal cancer, liver cancer, endometrial cancer, ovarian cancer, esophageal cancer, lung cancer, Kaposi's sarcoma, cervical cancer, pancreatic cancer, melanoma, prostate cancer, bladder cancer and leukemia.

13. The method according to claim 5, wherein the neoplastic disease is triple negative breast cancer.

14. (canceled)

15. (canceled)
